(19) United States
(12) Patent Application Publication
(10) Pub. No.: US 2025/0145922 A1
(43) Pub. Date: May 8, 2025

(54) MICROPHYSIOLOGICAL SYSTEM DEVICE

(71) Applicants: Sumitomo Riko Company Limited, Aichi (JP); Ginreilab Inc., Ishikawa (JP)

(72) Inventors: Satoshi TATSUMI, Komaki-shi (JP); Yasuhiro NAKAMURA, Komaki-shi (JP); Sayo NISHIIE, Komaki-shi (JP)

(73) Assignees: Sumitomo Riko Company Limited, Aichi (JP); Ginreilab Inc., Ishikawa (JP)

(21) Appl. No.: 19/015,521

(22) Filed: Jan. 9, 2025

Related U.S. Application Data
(63) Continuation of application No. PCT/JP2024/004801, filed on Feb. 13, 2024.

(30) Foreign Application Priority Data
Mar. 16, 2023 (JP) 2023-042394

(51) Int. Cl. C12M 1/32; C12M 1/00; C12M 1/34; C12M 3/00

(52) U.S. Cl. CPC C12M 23/12; C12M 23/38; C12M 23/44; C12M 41/00

(57) ABSTRACT

A microphysiological system device (1) includes: a container (2) in which a plurality of functional units (6a to 6d) is placed in a replaceable manner, and a channel member (3) disposed on a front side of the container (2) and defining at least part of a channel (33a, 33b) that causes the plurality of functional units (6a to 6d) placed in the container (2) to communicate with each other.

### CROSS-REFERENCE TO RELATED APPLICATION

This application is a continuation of PCT International Application No. PCT/JP2024/004801, filed on Feb. 13, 2024, which claims priority under 35 U.S.C § 119 (a) to Japanese Patent Application No. 2023-042394, filed on Mar. 16, 2023. Each of the above application(s) is hereby expressly incorporated by reference, in its entirety, into the present application.

### BACKGROUND

### Technical Field

The present disclosure relates to microphysiological system devices for use in drug discovery research etc.

### Background Art

In recent years, microphysiological system (MPS) devices have attracted attention as an alternative technology to animal testing. For example, WO2020/013851 discloses a fluidic device that includes a microplate with a plurality of interconnected wells. In the case of the fluidic device disclosed in this document, a manifold that distributes a fluid to the plurality of wells, a pump that pumps the fluid into the wells, etc. are disposed outside the fluidic device. These pieces of external equipment are connected to the wells via tubes.

In the case of the fluidic device disclosed in this document, an operator needs to accurately connect the plurality of wells to the external equipment having desired functions via tubes each time he or she rearranges the functions of the fluidic device. Moreover, it is necessary to remove the fluidic device from the tubes and carry the fluidic device to observation equipment (such as an inverted microscope) to observe a sample after an experiment. Since the fluidic device is mainly used inside an incubator, the configuration of this fluidic device makes the above work very difficult and complicated. As described above, conventional microphysiological systems have low usability. Therefore, a microphysiological system with high usability is desired.

### SUMMARY

According to an embodiment of the present disclosure, a microphysiological system device includes: a container in which a plurality of functional units is placed in a replaceable manner; and a channel member disposed on a front side of the container and defining at least part of a channel that causes the plurality of functional units placed in the container to communicate with each other.

### BRIEF DESCRIPTION OF THE DRAWINGS

FIG. 1 is a perspective view of a microphysiological system device of a first embodiment.

FIG. 2 is a transparent perspective view of the microphysiological system device.

FIG. 3 is a transparent top view of the microphysiological system device.

FIG. 4 is an exploded perspective view of the microphysiological system device.

FIG. 5 is an exploded perspective view of the microphysiological system device (excluding fastening members).

FIG. 6 is an exploded perspective view of a channel member of the microphysiological system device.

FIG. 7 is an enlarged view of the region VII in FIG. 6.

FIG. 8 is a sectional view in the direction VIII-VIII in FIG. 3.

FIG. 9 is an exploded perspective view of the channel member, container, and functional units of the microphysiological system device.

FIG. 10 is an exploded perspective view of the container and functional units of the microphysiological system device.

FIG. 11 is an exploded perspective view of a liquid feed unit (excluding a tray) of the microphysiological system device.

FIG. 12 is a sectional view in the direction XII-XII in FIG. 3.

FIG. 13 is a sectional view in the direction XIII-XIII in FIG. 3.

FIG. 14 is an exploded sectional view in a front-rear direction of a portion of the microphysiological system device (excluding the fastening members) in which the liquid feed unit is disposed.

FIG. 15 is an exploded sectional view in the front-rear direction of the portion of the microphysiological system device in which the liquid feed unit is disposed.

FIG. 16 is a sectional view in the front-rear direction of a portion of a microphysiological system device of a second embodiment in which a liquid feed unit is disposed.

FIG. 17 is a transparent partial top view of a microphysiological system device of a third embodiment.

FIG. 18 is a transparent top view of a microphysiological system device of a fourth embodiment.

FIG. 19 is a transparent top view of an MPS device of a fifth embodiment.

FIG. 20 is a front view of the MPS device.

FIG. 21 is a sectional view in the direction XXI-XXI in FIG. 19.

FIG. 22 is an enlarged view of the region XXII in FIG. 21.

FIG. 23 is a partial sectional view in the front-rear direction of a microphysiological system device of a further embodiment.

FIG. 24 is a schematic top view of a microphysiological system device of the present disclosure.

### MODES FOR CARRYING OUT THE INVENTION

(1) A microphysiological system device of the present disclosure is characterized by including: a container in which a plurality of functional units is placed in a replaceable manner; and a channel member disposed on a front side of the container and defining at least part of a channel that causes the plurality of functional units placed in the container to communicate with each other.

As used herein, the “plurality of functional units” includes a plurality of functional units of the same type and a plurality of functional units of different types. The “channel member defining at least part of the channel” includes a channel member that defines the entire channel and a channel member that defines only part of the channel.

In this configuration, the plurality of functional units placed in the container is replaceable. This allows to easily change the functions of the microphysiological system device by replacing the functional units as desired. Therefore, it is possible to mimic a wide variety of biological environments according to a desired organ, disease, etc. with higher reproducibility compared with conventional simple in vitro devices.

According to the microphysiological system device of the present disclosure, the plurality of functional units can be caused to communicate with each other through the channel is set in advance in the channel member. This can also reduce misconnection compared to the case where the plurality of functional units is manually caused to communicate with each other using a tube. Since the channel is defined by the channel member, the channel is less likely to deform than a tube. This allows to easily implement a desired flow rate and channel resistance.

According to the microphysiological system device of the present disclosure, equipment that has conventionally been handled as external equipment can be placed in the container as a constituent member of the functional unit. This can reduce the size of the microphysiological system device. Moreover, the plurality of functional units can be integrated. This improves handleability. As described above, the microphysiological system device of the present disclosure has high usability.

(1-1) It is preferable that at least one of the plurality of functional units placed in the container be a liquid feed unit including a balloon pump that is elastically expandable and contractible, and that the balloon pump be configured to feed a fluid stored therein to the channel by an elastic restoring force upon contraction. With this configuration, the fluid can be pumped using the elastic restoring force without a power supply. This improves handleability.

(2) In the above configuration (1), a size of the container is preferably equal to a size of a multi-well plate according to an industrial standard, as viewed in a front-back direction. As used herein, the “industrial standard” includes international standards (ISO, IEC, etc.) and national standards of each country (JIS, DIN, ANSI, BS, etc.). In this configuration, the size of the container is set to the size of a multi-well plate according to the industrial standard. Therefore, the container is highly versatile for use in equipment designed for this size.

(3) In any of the above configurations, the container preferably includes: a rectangular bottom wall including a pair of long-side portions disposed so as to face each other and a pair of short-side portions connecting the pair of long-side portions and disposed so as to face each other; a pair of side walls standing from the pair of long-side portions toward the front side; and a partition wall connecting the pair of side walls to each other, extending parallel to the short-side portion, and dividing a space between the pair of side walls into a plurality of container compartments, and the container compartment is preferably compatible with the plurality of functional units. In this configuration, the container compartment is compatible with the plurality of functional units. Therefore, it is possible to independently replace the functional unit by the container compartment.

(4) In any of the above configurations, the channel member preferably includes a plurality of contact portions disposed so as to face the plurality of functional units placed in the container, the contact portion preferably contacts the functional unit from the front side, and the channel is preferably open to a back surface of the contact portion. With this configuration, the functional units can be positioned with respect to the channel member by the contact portions. Moreover, the fluid can be caused to flow between the channel and the functional units via the contact portions.

(5) In any of the above configurations, the channel member preferably defines a plurality of the channels inside. With this configuration, the plurality of channels can be defined by the channel member alone without using any other members. Moreover, the relative positional relationship between or among the plurality of channels can be fixed.

(5-1) In any of the above configurations, the channel member preferably includes a flow rate control portion that controls a flow rate of the fluid flowing through a desired one of the channels. This configuration allows to control the flow rate of the fluid in the desired channel.

(5-2) In the above configuration (5-1), the flow rate control portion is preferably configured to adjust at least one of a shape in a channel length direction of the channel and a shape in a direction crossing (e.g., perpendicular to) the channel length direction of the channel. With this configuration, the flow rate can be easily controlled by adjusting the channel shape without using any other members.

(6) In any of the above configurations, the microphysiological system device preferably further includes a cover member disposed on a front side of the channel member and having higher rigidity than the channel member. With this configuration, the cover member can protect the channel member.

(6-1) In the above configuration (6), the microphysiological system device preferably further includes a fastening member that fastens the cover member and the container together. With this configuration, the cover member and the container can be combined by the fastening member.

(6-2) In the above configuration (6-1), the container and the channel member are preferably combined by the cover member and the fastening member after the functional unit is fixed by the channel member. With this configuration, the container and the channel member can be combined by the cover member and the fastening member. Moreover, variations in fastening force caused by differences in skills among operators can be reduced.

(6-3) In the above configuration (6-1) or (6-2), the channel member preferably includes a plurality of contact portions disposed so as to face the plurality of functional units placed in the container, the functional unit preferably has an opening that faces the contact portion, and the contact portion preferably includes a press-in portion that is pressed into the opening. With this configuration, the amount by which the press-in portion is pressed into the opening can be determined by fastening the cover member and the container together by the fastening member. Moreover, the functional unit can be positioned with respect to the channel member.

(7) In any of the above configurations (6-1) to (6-3), the cover member preferably includes a front-side engaging portion, the container preferably includes a back-side engaging portion, and the fastening member preferably engages with the front-side engaging portion and the back-side engaging portion. With this configuration, the fastening member can be attached to and detached from the cover member and the container by engaging and disengaging the fastening member with and from the front-side engaging portion and the back-side engaging portion. That is, the cover member, the channel member, and the container can be combined or separated.

(8) In the above configuration (7), the container preferably includes: a rectangular bottom wall including a pair of long-side portions disposed so as to face each other and a pair of short-side portions connecting the pair of long-side portions and disposed so as to face each other; a pair of side walls standing from the pair of long-side portions toward the front side; and a partition wall connecting the pair of side walls to each other, extending parallel to the short-side portion, and dividing a space between the pair of side walls into a plurality of container compartments. A size of the container is preferably equal to a size of a multi-well plate according to an industrial standard, as viewed in a front-back direction. The container compartment is preferably compatible with the plurality of functional units. The channel member preferably defines a plurality of the channels inside, and preferably includes: a flow rate control portion that controls a flow rate of a fluid flowing through a desired one of the channels; and a plurality of contact portions disposed so as to face the plurality of functional units placed in the container. The contact portion preferably contacts the functional unit from the front side, and the channel is preferably open to a back surface of the contact portion. The container, the channel member, and the cover member are preferably transparent. This configuration has the effects of the above configurations (1) to (7). The container, the channel member, and the cover member are transparent. This facilitates observation.

(9) In any of the above configurations, the container preferably includes a bottom wall and a side wall standing from the bottom wall toward the front side, and the side wall preferably has an opening extending through the side wall. With this configuration, the outside of the container and the inside of the container can be easily connected through the opening.

(10) In any of the above configurations, the container preferably includes a bottom wall, and the bottom wall preferably has an opening extending through the bottom wall. With this configuration, the functional unit can be easily positioned using the opening.

(11) In any of the above configurations, the channel member preferably includes a contact portion, the functional unit preferably has an opening, the contact portion preferably has a press-in portion that is pressed into the opening, and the press-in portion is preferably made of a material containing at least one of PDMS and resin.

With this configuration, the press-in portion of the contact portion of the channel member is pressed into the opening of the functional unit. The functional unit can therefore be easily positioned with respect to the channel member. Moreover, this configuration improves sealing performance between the channel member and the opening.

(11-1) In the above configuration (11), the channel member preferably includes a soft layer made of PDMS and a hard layer made of resin and having higher rigidity than the soft layer, in this order from front to back. The soft layer preferably includes a soft-layer protrusion protruding toward the back, and the hard layer preferably includes a tubular hard-layer protrusion protruding toward the back. The soft-layer protrusion is preferably located radially inside the hard-layer protrusion. A seal member made of an elastomer is preferably fitted on the outer peripheral surface of the hard-layer protrusion. The contact portion preferably includes the soft-layer protrusion, the hard-layer protrusion, and the seal member. The press-in portion preferably includes the seal portion and a portion of the contact portion that is located closer to the back side than the seal member. The seal member preferably elastically contacts the opening of the functional unit from inside in a radial direction.

With this configuration, the seal member is interposed between the hard-layer protrusion of the channel member and the opening of the functional unit. Elastic deformation of the seal member provides scaling (liquid-tightness, air-tightness, etc.) of the opening against the fluid.

(11-2) In the above configuration (11-1), the press-in portion preferably has a shape that becomes thinner toward its bottom. The functional unit is preferably a culture unit, and the culture unit preferably includes a culture vessel that has the opening and in which a culture medium is stored. A gas bubble storage space in which gas bubbles in the culture medium are stored is preferably defined immediately under the seal member inside the culture vessel.

With this configuration, the gas bubble storage space is defined immediately under the seal member, that is, near an upper end of an inner peripheral surface of a side wall of the culture vessel. Therefore, the gas bubbles in the culture medium can be moved upward and into the gas bubble storage space by the buoyancy of the gas bubbles themselves. This provides sufficient visibility during microscopic observation.

(12) In any of the above configurations, the channel is preferably used for degassing the functional unit. Gas bubbles may accidentally enter a functional unit for liquid (e.g., the culture unit that uses the culture medium). It is herein assumed that the microphysiological system device including a functional unit for liquid is used in an environment where a sudden pressure drop may occur (e.g., outer space etc.). In this case, the pressure drop may lead to larger gas bubbles in the liquid, which may result in the functional unit not working properly. In this regard, with the configuration, the functional unit can be degassed through the channel. This can reduce the possibility of the functional unit not working properly due to gas bubbles.

(12-1) In the above configuration (12), the channel preferably communicates with a gas bubble storage portion disposed inside or outside the container. With this configuration, gas bubbles in the culture medium can be stored in the gas bubble storage portion.

Disposing the gas bubble storage portion inside the container allows gas bubble management to be completed inside the microphysiological system device. This improves handleability compared to the case where the gas bubble storage portion is located outside the container.

Disposing the gas bubble storage portion outside the container increases design flexibility in size, shape, etc. of the gas bubble storage portion compared to the case where the gas bubble storage portion is disposed inside the container. This configuration can also reduce the impact of any trouble caused by gas bubbles on the microphysiological system device compared to the case where the gas bubble storage portion is disposed inside the container.

According to the microphysiological system device of the present disclosure, equipment that has conventionally been handled as external equipment can be placed in the container as a constituent member of the functional unit. This can reduce the size of the microphysiological system device. Moreover, the plurality of functional units can be integrated. This improves handleability. As described above, the microphysiological system device of the present disclosure has high usability. Furthermore, the functional units can be easily rearranged. It is also easy to reduce the size of the microphysiological system device, which improves workability. Since there is no need for complicated work such as manually causing the plurality of functional units to communicate with each other using tubes, contamination can be reduced.

Embodiments of a microphysiological system device of the present disclosure will now be described. Hereinafter, the microphysiological system device will be simply referred to as “MPS device” as appropriate.

### First Embodiment

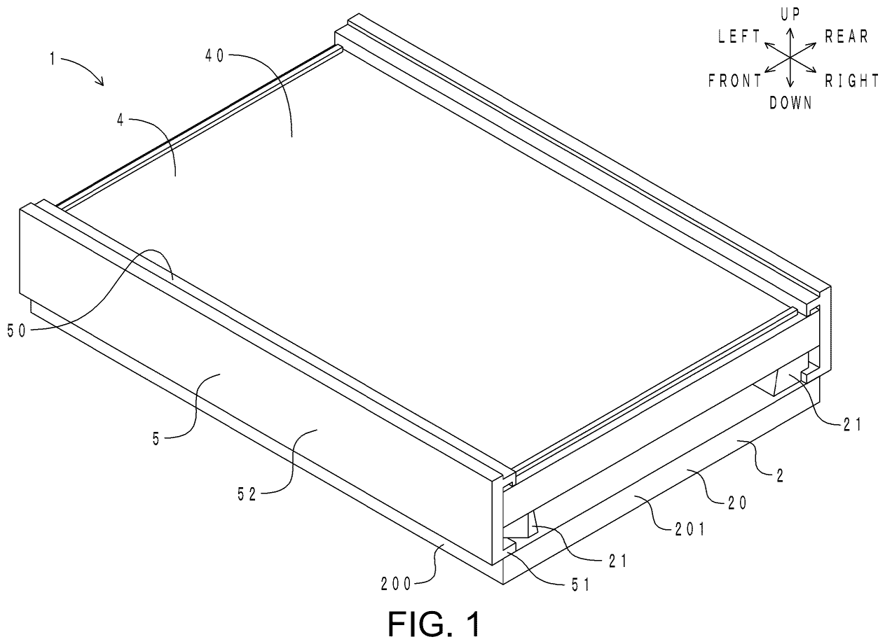

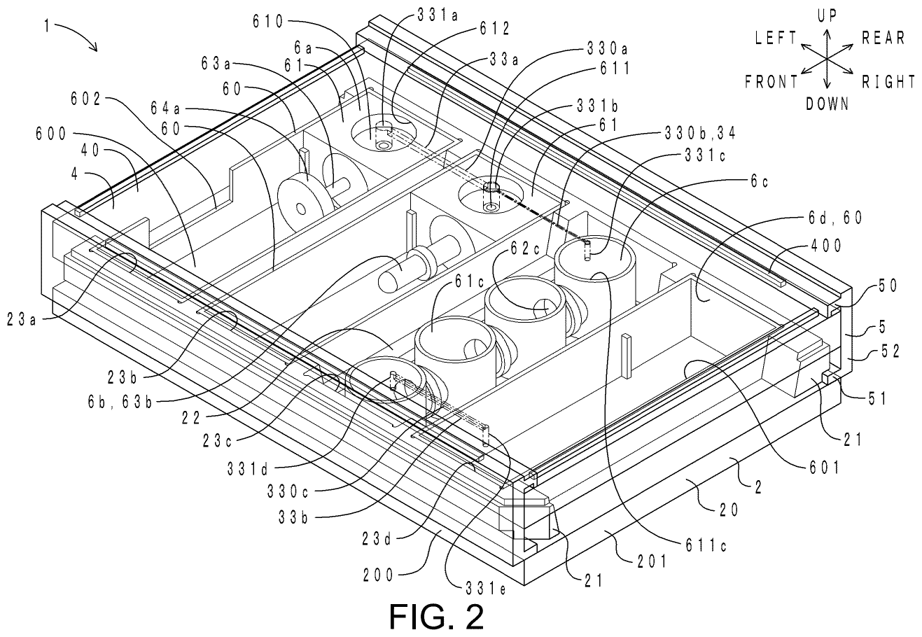

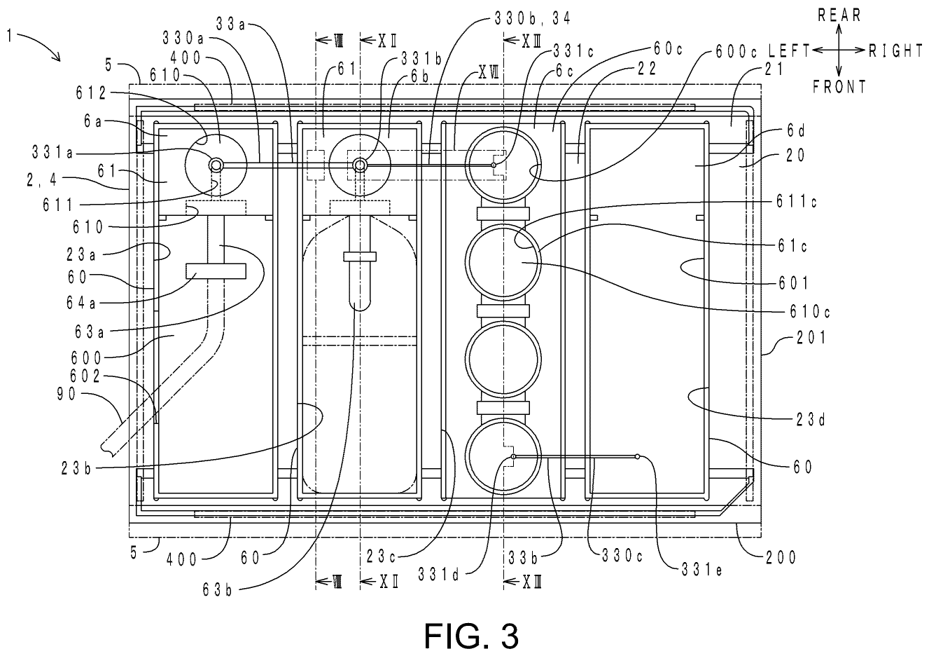

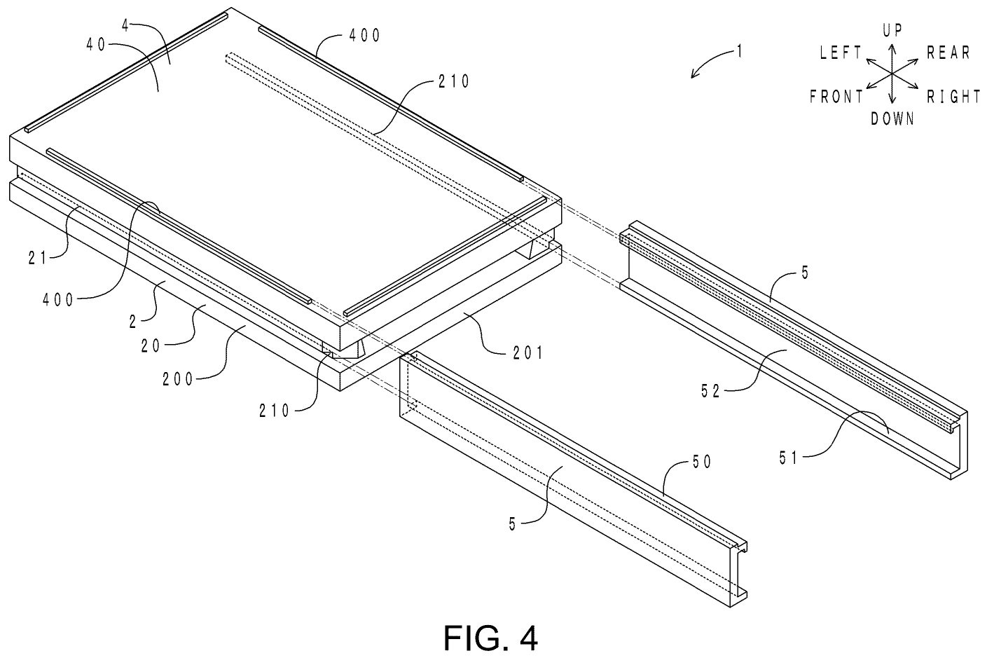

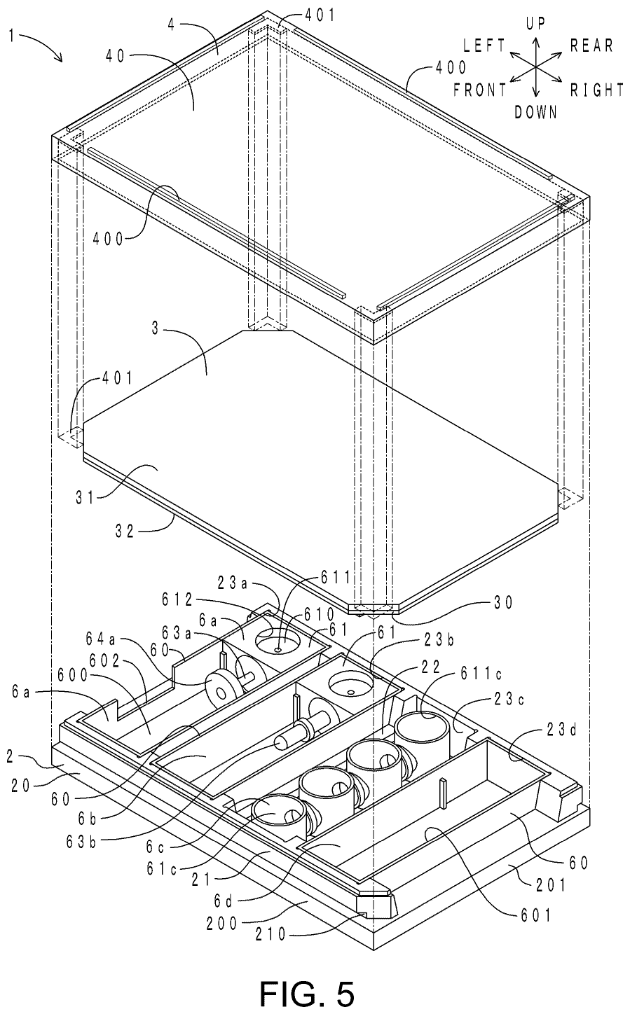

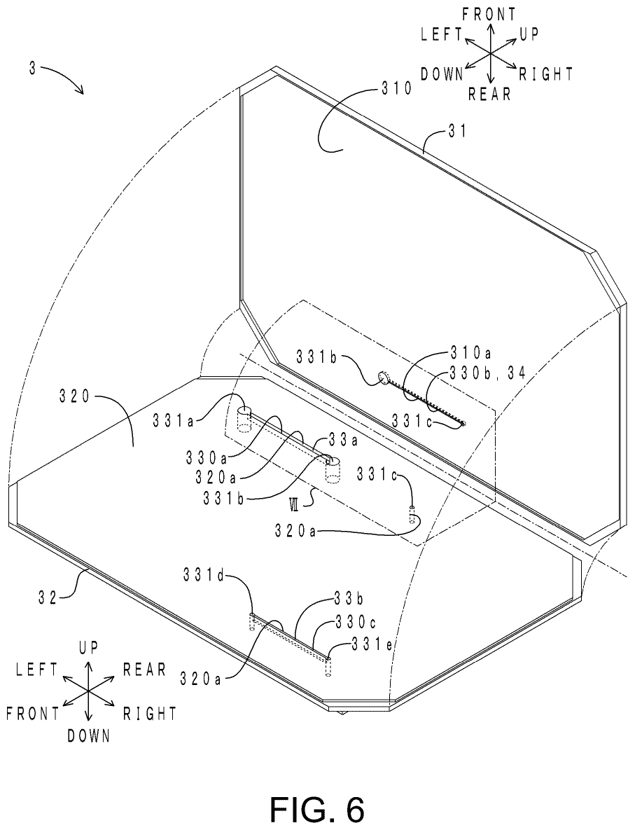

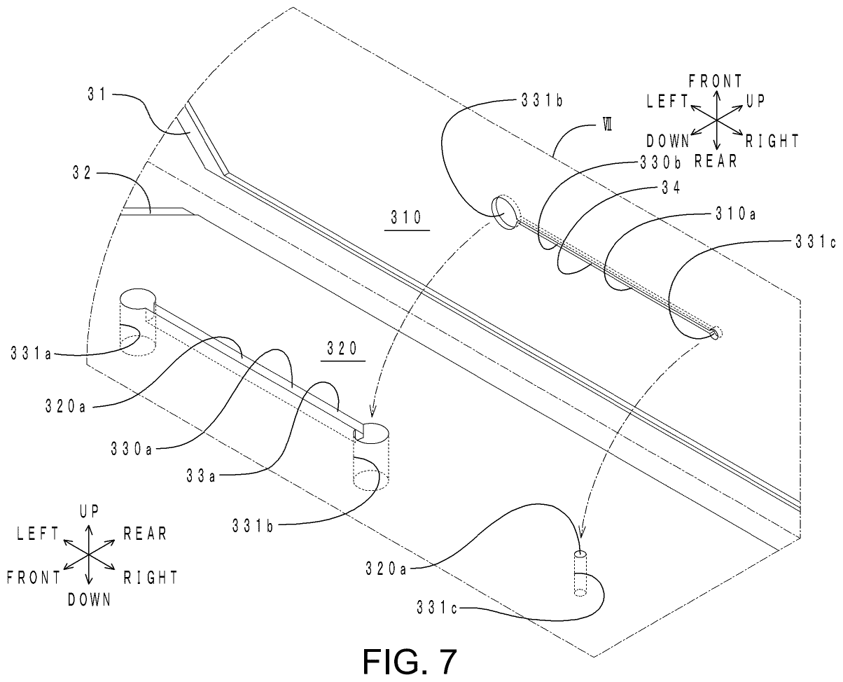

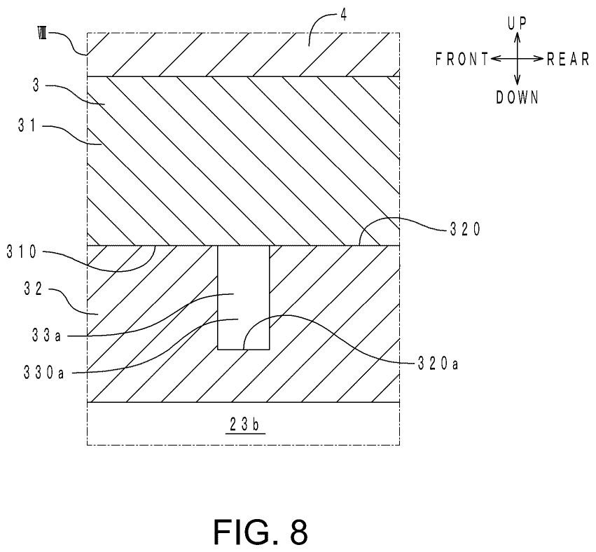

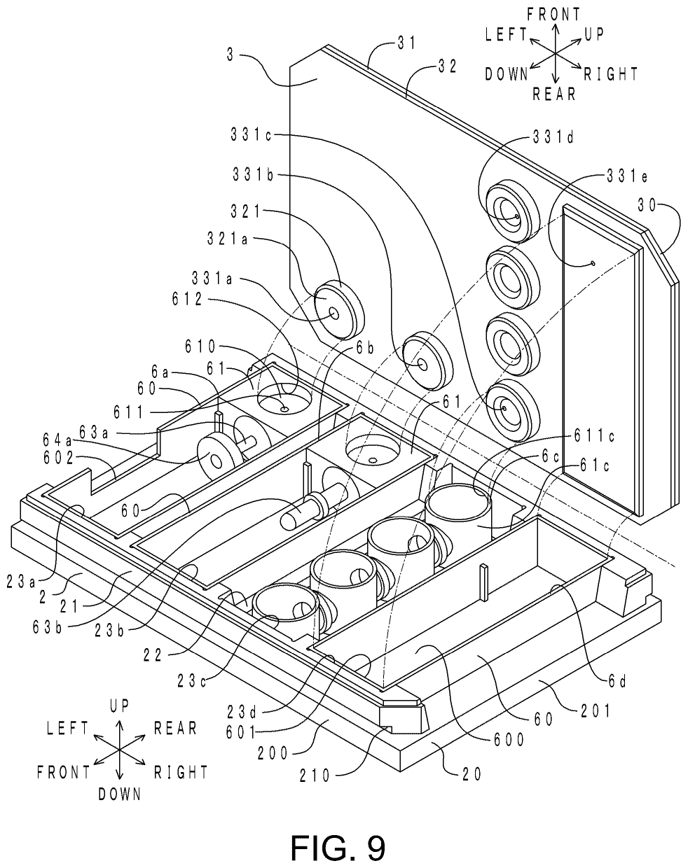

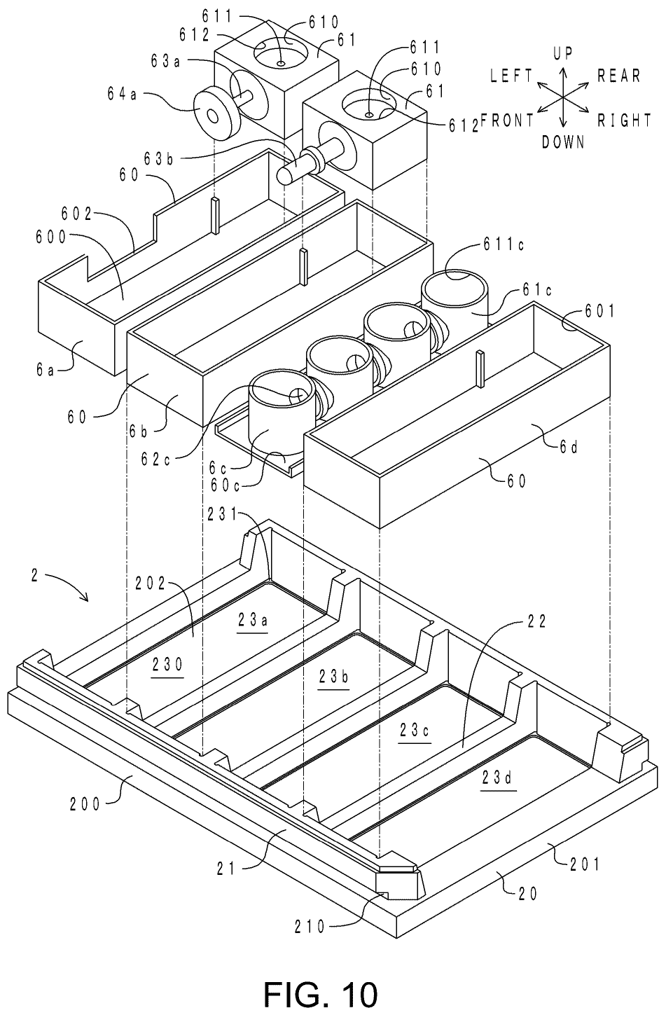

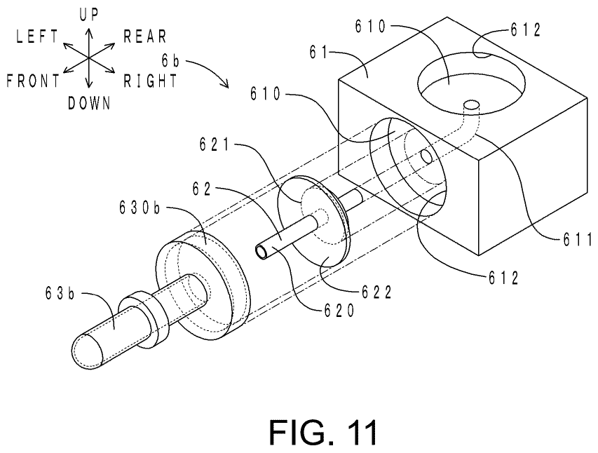

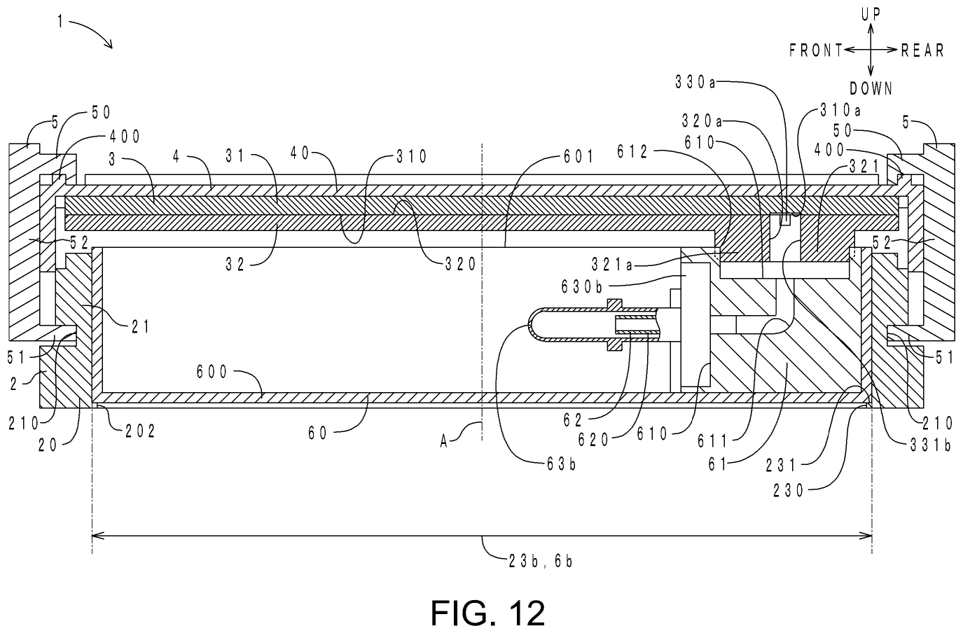

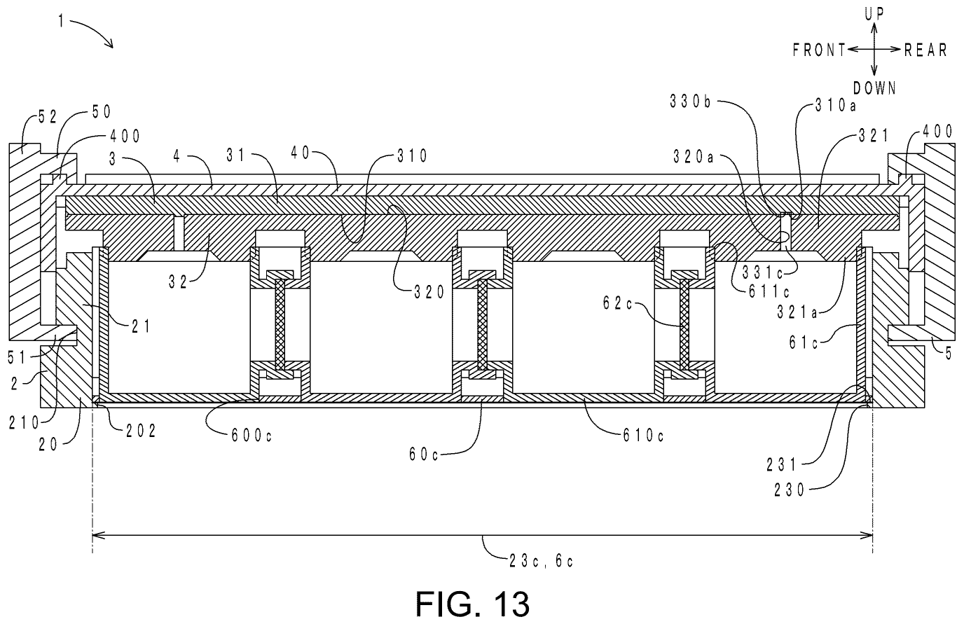

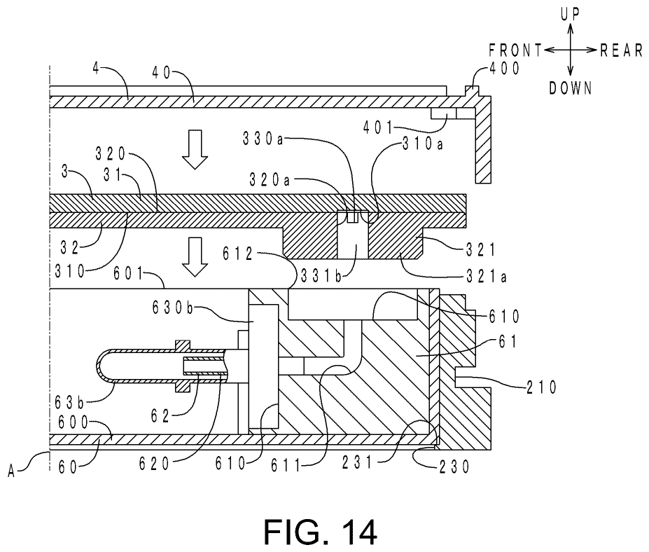

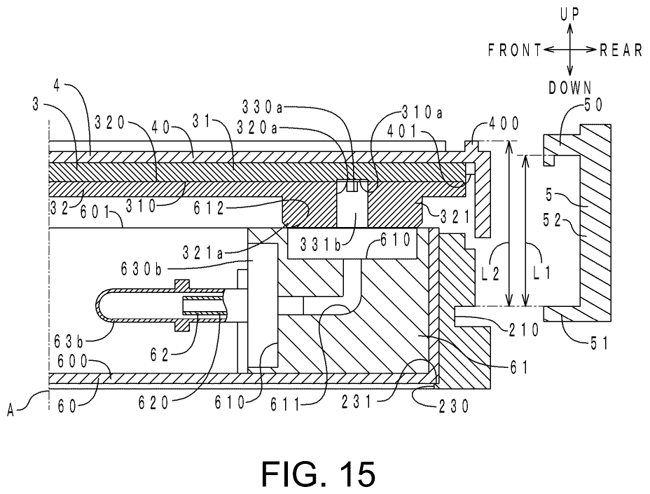

FIG. 1 is a perspective view of an MPS device of the present embodiment. FIG. 2 is a transparent perspective view of the MPS device. FIG. 3 is a transparent top view of the MPS device. FIG. 4 is an exploded perspective view of the MPS device. FIG. 5 is an exploded perspective view of the MPS device (excluding fastening members). FIG. 6 is an exploded perspective view of a channel member of the MPS device. FIG. 7 is an enlarged view of the region VII in FIG. 6. FIG. 8 is a sectional view in the direction VIII-VIII in FIG. 3. FIG. 9 is an exploded perspective view of the channel member, container, and functional units of the MPS device. FIG. 10 is an exploded perspective view of the container and functional units of the MPS device. FIG. 11 is an exploded perspective view of a liquid feed unit (excluding a tray) of the MPS device. FIG. 12 is a sectional view in the direction XII-XII in FIG. 3. FIG. 13 is a sectional view in the direction XIII-XIII in FIG. 3. FIG. 14 is an exploded sectional view in a front-rear direction of a portion of the MPS device (excluding the fastening members) in which the liquid feed unit is disposed. FIG. 15 is an exploded sectional view in the front-rear direction of the portion of the MPS device in which the liquid feed unit is disposed. In FIGS. 2 and 3, only channels 33a, 33b are shown regarding a channel member 3. In FIG. 3, fastening members 5 are shown by long dashed short dashed lines. The portion shown in FIGS. 14 and 15 corresponds to a portion on the rear side of a central axis (central axis in the front-rear direction of a MPS device 1) A shown in FIG. 12.

### [Configuration of MPS Device 1]

First, the configuration of the MPS device of the present embodiment will be described. As shown in FIGS. 1 to 5, 9, 10, 12, and 13, the MPS device 1 includes a container 2, a channel member 3, a cover member 4, fastening members 5, a supply unit 6a, a liquid feed unit 6b, a culture unit 6c, and a waste liquid storage unit 6d. The supply unit 6a, the liquid feed unit 6b, the culture unit 6c, and the waste liquid storage unit 6d are included in the concept of the “functional unit” in the present disclosure.

### (Container 2)

As shown in FIGS. 3 and 10, the functional units (supply unit 6a, liquid feed unit 6b, culture unit 6c, and waste liquid storage unit 6d) are disposed in the container 2 in a replaceable, addable/removable, and repositionable manner. The container 2 is transparent and made of polycarbonate. The size of the container 2 as viewed in an up-down direction (front-back direction) is equal to the size of a 96-well plate according to the ANSI/SBS standard. The ANSI/SBS standard is included in the concept of the “industry standard” in this disclosure. A 96-well plate is included in the concept of the “multi-well plate” in the present disclosure.

The container 2 includes a bottom wall 20, a pair of side walls 21, three partition walls 22, and container compartments 23a to 23d. The bottom wall 20 is in the form of a rectangular flat plate extending in a horizontal direction (front-rear and left-right directions, directions perpendicular to the up-down direction). The bottom wall 20 includes a pair of long-side portions 200 and a pair of short-side portions 201. The long-side portions 200 extend in the left-right direction. The pair of long-side portions 200 is disposed so as to face each other in the front-rear direction. The short-side portions 201 extend in the front-rear direction. The pair of short-side portions 201 is disposed so as to face each other in the left-right direction. The pair of short-side portions 201 connects the left ends and right ends of the pair of long-side portions 200 to each other. The bottom wall 20 has a rectangular opening 202.

The pair of side walls 21 stands upward from the pair of long-side portions 200. The side walls 21 are in the form of a rectangular plate. Recessed back-side guide portions 210 are provided on the outer surfaces of the side walls 21 (front surface of the front side wall 21 and rear surface of the rear side wall 21). The back-side guide portions 210 are included in the concept of the “back-side engaging portion” in the present disclosure. The back-side guide portions 210 extend in the left-right direction (same direction as front-side guide portions 400 described later).

Each of the three partition walls 22 connects the pair of side walls 21 to each other. The partition walls 22 are in the form of a rectangular plate. The partition walls 22 have a lower height in the up-down direction than the side walls 21. The partition walls 22 extend in the front-rear direction (same direction as the short-side portions 201). The three partition walls 22 divide the space between the pair of side walls 21 (space inside the container 2) and the opening 202 of the bottom wall 20 into the four container compartments 23a to 23d.

The four container compartments 23a to 23d have the same size as viewed in the up-down direction. The container compartments 23a to 23d are compatible with the plurality of functional units. The container compartments 23a to 23d extend in the front-rear direction. The four container compartments 23a to 23d are located next to each other in the left-right direction with the three partition walls 22 therebetween. Frame members 231 are disposed along the edges of openings 230 of the container compartments 23a to 23d (openings into which the opening 202 of the bottom wall 20 is divided by the partition walls 22).

### (Channel Member 3)

As shown in FIGS. 5, 9, 12 and 13, the channel member 3 fixes the functional units (supply unit 6a, liquid feed unit 6b, culture unit 6c, and waste liquid storage unit 6d). As shown in FIG. 9, the channel member 3 seal openings 601, 611c, and 612 of the functional units (specifically, as shown in FIG. 9, opening 601 of a tray 60 of the waste liquid storage unit 6d, openings 611c of four culture vessels 61c of the culture unit 6c, upper opening 612 of an import member 61 of the supply unit 6a, and upper opening 612 of an import member 61 of the liquid feed unit 6b; the same applies below) in a liquid-tight manner. The channel member 3 is transparent and made of PDMS (polydimethylsiloxane, a type of silicone, an elastomer). The channel member 3 has rubber elasticity. The channel member 3 is in the form of a rectangular plate extending in the horizontal direction. The channel member 3 has chamfered portions 30 at its four corners. The channel member 3 is disposed on the upper side (front side) of the container 2.

As shown in FIGS. 5 to 8, the channel member 3 includes a first layer (front layer member) 31, a second layer (back layer member) 32, and two channels 33a, 33b. A first-layer recessed portion 310 is formed in the lower surface of the first layer 31. A first recess 310a is formed in the lower surface of the first-layer recessed portion 310. The second layer 32 is placed on the lower side (back side) of the first layer 31. A second-layer raised portion 320 protrudes from the upper surface of the second layer 32. Three second recesses 320a are formed in the upper surface of the second-layer raised portion 320. The second-layer raised portion 320 and the first-layer recessed portion 310 overlap each other as viewed in the up-down direction. The second-layer raised portion 320 is inserted in the first-layer recessed portion 310.

As shown in FIG. 9, seven contact portions 321 protrude from the lower surface of the second layer 32. The contact portions 321 are disposed so as to face the functional units placed in the container 2. The contact portions 321 contacts the functional units from above. Press-in portions 321a are set at the lower ends (distal ends) of the contact portions 321. The press-in portions 321a have a tapered shape that becomes thinner toward its bottom. At least part of each press-in portion 321a is pressed into the openings 601, 611c, and 612 of the functional units.

As shown in FIGS. 3 and 5 to 8, the two channels 33a, 33b are defined inside the channel member 3. The channels 33a, 33b allow the plurality of functional units placed in the container 2 to communicate with each other. The channels 33a, 33b are composed of horizontally extending portions (portions extending in the horizontal direction (layer direction (direction in which the first layer 31 and the second layer 32 extend))) 330a to 330c and vertically extending portions (portions extending in the front-back direction) 331a to 331e. The horizontally extending portions 330a, 330c are formed in clearance between each of the second recesses 320a and the lower surface of the first-layer recessed portion 310. The horizontally extending portion 330b is formed in clearance between the first recess 310a and the upper surface of the second-layer raised portion 320. That is, the horizontally extending portions 330a, 330c are formed at the boundary between the first layer 31 and the second layer 32. The horizontally extending portions 330a to 330c extend in the horizontal direction. The vertically extending portions 331a, 331d, and 331c are located in the second layer 32. The vertically extending portions 331b, 331c are each located in both the first layer 31 and the second layer 32. The vertically extending portions 331a to 331c extend in the up-down direction. The upper ends of the vertically extending portions 331a to 331c are continuous with the horizontally extending portions 330a to 330c. The lower ends of the vertically extending portions 331a to 331e are open to the lower surfaces of the contact portions 321 (press-in portions 321a).

As shown in FIGS. 2 and 3, the channel 33a extends across the three adjacent container compartments 23a to 23c. The channel 33a includes the two horizontally extending portions 330a, 330b and the three vertically extending portions 331a to 331c. The vertically extending portion 331a is open to the container compartment 23a, the vertically extending portion 331b is open to the container compartment 23b, and the vertically extending portion 331c is open to the container compartment 23c. The horizontally extending portion 330a connects the vertically extending portion 331a and the vertically extending portion 331b. The horizontally extending portion 330b connects the vertically extending portion 331b and the vertically extending portion 331c.

As shown in FIGS. 2 and 3, the channel 33b extends across the two adjacent container compartments 23c, 23d. The channel 33b includes the one horizontally extending portion 330c and the two vertically extending portions 331d, 331e. The vertically extending portion 331d is open to the container compartment 23c, and the vertically extending portion 331e is open to the container compartment 23d. The horizontally extending portion 330c connects the vertically extending portion 331d and the vertically extending portion 331c.

As shown in FIGS. 3 and 7, a flow rate control portion 34 controls the flow rate of a culture medium flowing through the channel 33a. The culture medium is included in the concept of the “fluid” in the present disclosure. The flow rate control portion 34 is the horizontally extending portion 330b. The horizontally extending portion 330b has a smaller sectional channel area than the vertically extending portion 331b. The horizontally extending portion 330b and the vertically extending portion 331b are different in channel wall resistance and channel shape. The horizontally extending portion 330b, that is, the flow rate control portion 34, controls the flow rate of the culture medium by the sectional channel area, the channel wall resistance, the channel shape, etc.

### (Cover Member 4)

As shown in FIGS. 5, 12, and 13, the cover member 4 reinforces the channel member 3. The cover member 4 is disposed on the upper side of the channel member 3. The cover member 4 presses the channel member 3 from above. The cover member 4 is transparent and made of polystyrene. The cover member 4 has higher rigidity than the channel member 3. The cover member 4 is in the form of a rectangular box that is open downward. A top wall 40 of the cover member 4 is in the form of a rectangular flat plate extending in the horizontal direction. The upper surface of the top wall 40 is a smooth flat surface. A pair of front-side guide portions 400 protrudes from the upper surface of the top wall 40. The front-side guide portions 400 are included in the concept of the “front-side engaging portion” in the present disclosure. The front-side guide portions 400 extend in the horizontal direction (left-right direction). The pair of front-side guide portions 400 is disposed along both front and rear edges of the upper surface of the top wall 40. Four positioning portions 401 protrude from the lower surface of the top wall 40. The positioning portions 401 have an L-shape as viewed in the up-down direction. The four positioning portions 401 are disposed at the four corners of the lower surface of the top wall 40. The four positioning portions 401 are disposed horizontally outside the chamfered portions 30 at the four corners of the channel member 3.

### (Fastening Members 5)

As shown in FIGS. 1 to 4, 12, 13, and 15, the pair of fastening members 5 fixes the cover member 4 to the container 2 containing the functional units with the channel member 3 sandwiched between the cover member 4 and the container 2. As shown in FIG. 3, the fastening members 5 are disposed along both front and rear edges of the MPS device 1 as viewed in the up-down direction. The fastening members 5 are disposed so as to avoid the culture unit 6c (portion to be observed) as viewed in the up-down direction. The fastening members 5 are made of polycarbonate and extend in the horizontal direction (left-right direction).

The rear fastening member 5 will be described below as a representative of the pair of front and rear fastening members 5. As shown in FIGS. 12 and 15, the rear fastening member 5 has a C-shape that is open forward (inward) as viewed in the left-right direction (direction in which the fastening member 5 extends). The fastening member 5 includes a front-side guided portion 50, a back-side guided portion 51, and a connecting portion 52. The front-side guided portion 50 protrudes forward from the upper edge of the connecting portion 52. The front-side guided portion 50 is in pressure contact with the front-side guide portion 400 from above. The front-side guided portion 50 engages with the front-side guide portion 400 from front (inside). The front-side guided portion 50 is slidable in the left-right direction with respect to the front-side guide portion 400. The back-side guided portion 51 protrudes forward from the lower edge of the connecting portion 52. The back-side guided portion 51 is in pressure contact with the back-side guide portion 210 from below. The back-side guided portion 51 is slidable in the left-right direction with respect to the back-side guide portion 210. The cover member 4, the channel member 3, and the container 2 are fixed together in the up-down direction by the front-side guided portion 50 and the back-side guided portion 51. The front fastening member 5 has the same configuration as the rear fastening member 5. The front fastening member 5 is disposed symmetrically with the rear fastening member 5 in the front-rear direction.

### (Functional Units)

As shown in FIG. 10, the container 2 is divided into the four container compartments 23a to 23d located next to each other in the left-right direction. The supply unit 6a, the liquid feed unit 6b, the culture unit 6c, and the waste liquid storage unit 6d are detachably placed in the container compartment 23a, the container compartment 23b, the container compartment 23c, and the container compartment 23d, respectively.

### (Supply Unit 6a)

The culture medium is supplied to the supply unit 6a from outside the MPS device 1. As shown in FIG. 10, the supply unit 6a includes a tray 60, an import member 61, a connector 62 (see FIG. 11), a tube 63a, and a check valve 64a. The tray 60 is transparent and made of polycarbonate. The tray 60 is in the form of a rectangular box that is open upward. The tray 60 is detachably placed in the container compartment 23a. In the installed state, a bottom wall 600 of the tray 60 rests on the frame member 231 in the opening 230 of the container compartment 23a. The tray 60 has an opening 602 in its left (outer) side wall.

The import member 61 has the shape of a rectangular parallelepiped. The import member 61 is detachably placed in a rear end portion of the space inside the tray 60. The import member 61 has recesses 610 in its upper and front surfaces. The two recesses 610 communicate with each other via a communication hole 611 extending in an L-shape. As shown in FIG. 9, the press-in portion 321a of the contact portion 321 of the channel member 3 is pressed into the opening 612 of the recess 610 in the upper surface. The communication hole 611 of the import member 61 is connected to the channel 33a (vertically extending portion 331a) via the press-in portion 321a.

The connector is disposed in the recess 610 in the front surface. The connector connects the communication hole 611 of the import member 61 to the tube 63a. The configuration of the connector will be described later (see FIG. 11). The tube 63a can be attached to and detached from the import member 61 via the connector. The check valve 64a is connected to the tube 63a. As shown in FIG. 3, a tube 90 connected to an infusion pump (such as a syringe pump) can be connected to the check valve 64a from outside the MPS device 1. The check valve 64a allows the culture medium to flow only in the direction from the infusion pump toward the import member 61. As shown in FIG. 3, the tube 90 can be inserted into and removed from the tray 60 through the opening 602.

### (Liquid Feed Unit 6b)

The culture medium is supplied from the supply unit 6a to the liquid feed unit 6b. The liquid feed unit 6b can store the supplied culture medium. The liquid feed unit 6b can slowly pump the culture liquid stored therein into the culture unit 6c. As shown in FIGS. 10 to 12, the liquid feed unit 6b includes a tray 60, an import member 61, a connector 62, and a balloon pump 63b. The tray 60 is the same as the tray 60 of the supply unit 6a (except the opening 602), the import member 61 is the same as the import member 61 of the supply unit 6a, and the connector 62 is the same as the connector of the supply unit 6a.

As shown in FIG. 9, the press-in portion 321a of the contact portion 321 of the channel member 3 is pressed into the opening 612 of the recess 610 in the upper surface of the import member 61. The communication hole 611 of the import member 61 is connected to the channel 33a (vertically extending portion 331b) via the press-in portion 321a.

The balloon pump 63b is connected to the communication hole 611 of the import member 61 via the connector 62. The balloon pump 63b is made of PDMS and has rubber elasticity. As shown by long dashed short dashed lines in FIG. 3, the balloon pump 63b is expandable in the tray 60.

As shown in FIG. 11, the connector 62 includes a tube member 620, an inner flange 621, and an outer flange 622. The connector 62 has a luer-lock mechanism according to ISO 80369-7. The inner flange 621 and the outer flange 622 are fitted on the tube member 620. The inner flange 621 is disposed on the rear side of the outer flange 622. The inner flange 621 is placed in the front recess 610 of the import member 61. A portion of the tube member 620 that is located rearward of the inner flange 621 is press-fitted in the communication hole 611. A hollow flange 630b is disposed at the rear end of the balloon pump 63b. The hollow flange 630b has a bottomed cylindrical shape that is open rearward. The hollow flange 630b has in its front wall (bottom wall) a through hole communicating with the inside of the balloon pump 63b. The hollow flange 630b has thread grooves (not shown) in its inner peripheral surface. The outer flange 622 is threadedly engaged with the thread grooves. A portion of the tube member 620 that is located forward of the outer flange 622 is inserted in the balloon pump 63b.

### (Culture Unit 6c)

The culture medium is supplied from the liquid feed unit 6b to the culture unit 6c. The culture unit 6c cultures cells. As shown in FIGS. 10 and 13, the culture unit 6c includes a base 60c, four culture vessels 61c, and three filters 62c. The base 60c has four fixing holes 600c (same number as the maximum number of culture vessels 61c that can be placed). The culture vessels 61c are transparent and made of polystyrene. The culture vessels 61c have a bottomed cylindrical shape that is open upward. Bottom walls 610c of the culture vessels 61c are fitted in the fixing holes 600c. The number of culture vessels 61c can be changed (increased or decreased) as desired within the range of the number of fixing holes 600c. The positions of the culture vessels 61c can be changed as desired according to the positions of the fixing holes 600c. The lower surfaces of the bottom walls 610c are smooth flat surfaces. The culture vessels 61c contain cells. The four culture vessels 61c are connected to each other in the front-rear direction. The filters 62c are made of polycarbonate and are each disposed between a pair of adjacent culture vessels 61c with a seal (not shown) therebetween. The filters 62c selectively allows only a predetermined substance (e.g., a signal messenger (exosomes etc.)) to pass therethrough. As shown in FIG. 9, the press-in portions 321a of the contact portions 321 of the channel member 3 are pressed into the openings 611c of the culture vessels 61c. Of the four culture vessels 61c, the rearmost culture vessel 61c is connected to the channel 33a (vertically extending portion 331c) via the press-in portion 321a. The culture medium is introduced into the rearmost culture vessel 61c from the balloon pump 63b through the channel 33a. Of the four culture vessels 61c, the frontmost culture vessel 61c is connected to the channel 33b (vertically extending portion 331d) via the press-in portion 321a.

### (Waste Liquid Storage Unit 6d)

The waste liquid storage unit 6d stores waste liquid after an experiment. As shown in FIG. 10, the waste liquid storage unit 6d includes a tray 60. The tray 60 is the same as the tray 60 of the liquid feed unit 6b. As shown in FIG. 9, the press-in portion 321a of the contact portion 321 of the channel member 3 is pressed into the opening 601 of the tray 60. The tray 60 is connected to the channel 33b (vertically extending portion 331c) via the press-in portion 321a.

### [Method for Assembling MPS Device 1]

Next, a method for assembling the MPS device 1 of the present embodiment will be described. The assembly method includes a placing step, a press-in step, a fluid supply step, and a fastening step. In the placing step, desired functional units are placed in the container 2. That is, as shown in FIG. 10, desired functional units (in this example, supply unit 6a, liquid feed unit 6b, culture unit 6c, and waste liquid storage unit 6d) are first assembled outside the container 2. Next, the functional units are installed in the container 2. Specifically, the supply unit 6a is installed in the container compartment 23a, the liquid feed unit 6b is installed in the container compartment 23b, the culture unit 6c is installed in the container compartment 23c, and the waste liquid storage unit 6d is installed in the container compartment 23d. As shown by long dashed short dashed lines in FIG. 3, the tube 90 connected to an infusion pump is connected to the check valve 64a of the supply unit 6a.

In the press-in step, the channel member 3 is attached to the functional units. That is, as shown in FIGS. 9 and 14, the channel member 3 is first brought close to the functional units (supply unit 6a, liquid feed unit 6b, culture unit 6c, waste liquid storage unit 6d) from above. Next, the openings 601, 611c, and 612 of the functional units are sealed by the contact portions 321 of the channel member 3. Specifically, the press-in portions 321a are pressed into the openings 601, 611c, and 612.

In the fluid supply step, the culture medium is supplied to the balloon pump 63b. That is, as shown in FIG. 3, the culture medium is supplied from the infusion pump to the balloon pump 63b via the check valve 64a, the tube 63a, the communication hole 611 of the import member 61 of the supply unit 6a, the channel 33a, and the communication hole 611 of the import member 61 of the liquid feed unit 6b. As shown by long dashed short dashed lines in FIG. 3, as the culture medium is supplied to the balloon pump 63b, the balloon pump 63b expands while accumulating clastic energy.

As shown in FIGS. 3 and 7, the horizontally extending portion 330b has a smaller sectional channel area than the vertically extending portion 331b. The horizontally extending portion 330b therefore has a higher channel resistance than the vertically extending portion 331b. Accordingly, the culture medium flowing out of the horizontally extending portion 330a flow preferentially into the vertically extending portion 331b over into the horizontally extending portion 330b. As shown by long dashed short dashed lines in FIG. 3, the culture medium thus flows preferentially into the balloon pump 63b. After the supply of the culture medium is completed, the tube 90 is removed from the check valve 64a via the opening 602. The check valve 64a prevents backflow of the culture medium.

In the fastening step, the cover member 4 is attached to the container 2 by the pair of fastening members 5. That is, as shown in FIG. 14, the channel member 3 is first pressed down from above with the cover member 4. Next, as shown in FIG. 4, the pair of fastening members 5 is attached to the cover member 4 and the container 2. Specifically, the front-side guided portion 50 is caused to slide on the front-side guide portion 400. The back-side guided portion 51 is also caused to slide on the back-side guide portion 210. The fastening members 5 reduce deformation of the cover member 4. The cover member 4 positions the channel member 3 with respect to the openings 601, 611c, and 612 of the functional units. The cover member 4 fixes the amount by which the press-in portions 321a are pressed into the openings 601, 611c, and 612. The MPS device 1 is assembled in this manner.

### [Operation of MPS Device 1]

Next, the operation of the MPS device 1 of the present embodiment will be described. The assembled MPS device 1 (in the state shown in FIG. 1) is placed in an incubator with a predetermined atmosphere (e.g., temperature: 37° C., humidity: 95%, air composition: 5 to 20% of O₂, 5 to 10% of CO₂, etc.). As shown in FIGS. 2 and 3, the culture medium stored in the balloon pump 63b is discharged by the elastic energy accumulated in the balloon pump 63b. The check valve 64a prevents backflow of the culture medium. The discharged culture medium therefore flows preferentially into the horizontally extending portion 330b. The horizontally extending portion 330b has a smaller sectional channel area than the horizontally extending portion 330a. The flow rate of the culture medium is therefore restricted in the horizontally extending portion 330b. Accordingly, the culture medium slowly flows through the horizontally extending portion 330b over a long period of time. The culture medium flows into the rearmost culture vessel 61c through the horizontally extending portion 330b and the vertically extending portion 331c. The culture medium passes alternately through the four culture vessels 61c and the three filters 62c from rear to front. The culture medium after the passage, that is, waste liquid, flows from the frontmost culture vessel 61c through the channel 33b into the tray 60 of the waste liquid storage unit 6d.

When observing cells, an operator manually transports the MPS device 1 from the incubator to the stage of an inverted fluorescence microscope. The MPS device 1 is transparent as a whole. The pair of fastening members 5 is disposed at both front and rear edges. This allows to easily observe the culture unit 6c, namely the portion to be observed, without opening the cover member 4. The culture unit 6c, namely the portion to be observed, can be easily observed even while the MPS device 1 is contained in the incubator.

### [Functions and Effects]

Next, functions and effects of the MPS device of the present embodiment will be described. As shown in FIG. 10, the plurality of functional units (in this example, supply unit 6a, liquid feed unit 6b, culture unit 6c, and waste liquid storage unit 6d) placed in the container 2 is replaceable. This allows to easily change the functions of the MPS device 1 by replacing the functional units as desired. Therefore, it is possible to mimic a wide variety of biological environments according to a desired organ, disease, etc. with higher reproducibility compared with conventional simple in vitro devices.

As shown in FIG. 6, the channels 33a, 33b are defined in advance in the channel member 3 according to the functional units to be placed in the container 2. This allows the plurality of functional units to easily communicate with each other. This can also reduce misconnection compared to the case where the plurality of functional units is manually caused to communicate with each other using tubes. Since the channels 33a, 33b are defined by the channel member 3, the channels 33a, 33b are less likely to deform than tubes. This allows to easily implement a desired flow rate and channel resistance. Since there is no need to manually cause the plurality of functional units to communicate with each other using tubes, viruses, bacteria, dust, etc. are less likely to enter the functional units. This makes it easier to ensure cleanliness of the functional units. Contamination is also less likely to occur.

As shown in FIGS. 6 to 8, the channel member 3 has a two-layer structure consisting of the first layer 31 and the second layer 32. Therefore, the boundary between the first layer 31 and the second layer 32 can be used to define the channels 33a, 33b. The second-layer raised portion 320 of the second layer 32 is fitted in the first-layer recessed portion 310 of the first layer 31. The first layer 31 and the second layer 32 can thus be easily positioned. The channels 33a, 33b are defined inside the channel member 3. Therefore, the channels 33a, 33b can be defined without using any other members. Moreover, the relative positional relationship between the channels 33a, 33b can be fixed.

As shown in FIGS. 9, 12 and 13, the contact portions 321 contact the functional units from above. The channels 33a, 33b are open to the lower surfaces of the contact portions 321 (press-in portions 321a). The functional units can therefore be positioned with respect to the channel member 3 by the contact portions 321. Moreover, the culture medium can be caused to flow between each channel 33a, 33b and the functional units via the contact portions 321.

As shown in FIGS. 3 and 7, the channel member 3 includes the flow rate control portion 34. The flow rate control portion 34 changes the sectional channel shape of the horizontally extending portion 330b of the channel 33a (shape in a direction perpendicular to the channel length direction of the channel 33a) from the sectional channel shape of the horizontally extending portion 330a. This allows the flow rate of the culture medium in the horizontally extending portion 330b to be adjusted without using an external control unit, power source, wire, etc.

As shown in FIG. 7, the first layer 31 has the recessed horizontally extending portion 330b (flow rate control portion 34), and the second layer 32 has the recessed horizontally extending portion 330a. That is, a narrow groove (horizontally extending portion 330b) and a wide groove (horizontally extending portion 330a) are formed in different layers. This facilitates grooving compared to the case where a plurality of grooves (horizontally extending portion 330a, 330b) with different widths and depths is formed in a single layer (grooves in this case are also included in the concept of the horizontally extending portion in the present disclosure).

According to the MPS device 1 of the present embodiment, equipment that has conventionally been handled as external equipment (e.g., a pump (balloon pump 63b shown in FIG. 5)) can be placed in the container 2 as constituent members of the functional units. This can reduce the size of the MPS device 1. Moreover, the plurality of functional units can be integrated. This improves handleability. As described above, the MPS device 1 of the present embodiment has high usability.

The size of the container 2 shown in FIG. 3 is set to the size of a 96-well plate according to the ANSI/SBS standard. The container 2 is therefore highly versatile for use in equipment designed for this size (incubators, analytical equipment (microscopes etc.)).

As shown in FIG. 10, the container compartments 23a to 23d have the same size. The container compartments 23a to 23d are compatible with the plurality of functional units. Therefore, it is possible to independently replace the functional unit by the container compartment 23a to 23d.

As shown in FIG. 10, the same trays 60 are used for the plurality of functional units. This can reduce the manufacturing cost of the trays 60 and therefore the MPS device 1 compared to the case where each tray 60 is a product exclusively for a specific functional unit. This also facilitates rearrangement of the functional units. The same applies to the import members 61 and the connectors 62.

As shown in FIG. 10, the bottom wall 20 of the container 2 has the opening 202. This makes it easier to adjust the focal length of an inverted fluorescence microscope compared to the case where there is no opening 202 (case where the bottom wall 20 is solid).

The cover member 4 shown in FIGS. 12 and 13 has higher rigidity than the channel member 3. Therefore, the channel member 3 can be protected. The container 2 and the channel member 3 can be combined by the cover member 4 and the fastening members 5. Moreover, variations in fastening force caused by differences in skills among operators can be reduced.

As shown in FIG. 15, L1 represents the distance between the lower surface of the front-side guided portion 50 and the upper surface of the back-side guided portion 51. L2 represents the distance between the upper surface of the front-side guide portion 400 and the upper surface of the back-side guide portion 210 when the lower surface of the press-in portion 321a is flush with the opening 612 (when the press-in portion 321a has not been pressed into the opening 612). The distance L1 is smaller than the distance L2. The amount by which the press-in portions 321a are pressed into the openings 601, 611c, and 612 of the functional units can be determined by fastening the cover member 4 and the container 2 together by the fastening members 5. The press-in portions 321a have a tapered shape that becomes thinner toward its bottom. Therefore, part of a downward pressing force applied from the channel member 3 to the functional units acts on the functional units in the horizontal direction. The functional units can thus be positioned with respect to the channel member 3 in the horizontal direction. Since the press-in portions 321a are pressed into the openings 601, 611c, and 612, this provides high sealing performance between the channels 33a, 33b and the openings 601, 611c, and 612. Contamination is also less likely to occur.

As shown in FIG. 4, the fastening members 5 can be attached to and detached from the cover member 4 and the container 2 by sliding in the left-right direction. This allows the cover member 4, the channel member 3, and the container 2 to be easily combined and separated from each other.

As shown in FIGS. 1 to 3, the fastening members 5 are disposed along the entire length in the left-right direction (longitudinal direction) of both front and rear edges of the cover member 4. This can reduce deflection of the cover member 4 along its entire length in the longitudinal direction.

As shown in FIGS. 2 and 3, portions of the cover member 4, channel member 3, and container 2 that overlap the culture unit 6c as viewed in the up-down direction are all transparent. This allows the culture unit 6c to observed from the outside in the up-down direction of the MPS device 1. The fastening members 5 do not overlap the culture unit 6c as viewed in the up-down direction. This can reduce the possibility of the fastening members 5 interfering with observation.

Both a portion of the upper surface of the top wall 40 of the cover member 4 that overlaps the culture unit 6c and the lower surface of the bottom wall 610c of the culture vessel 61c are smooth and flat. Therefore, light is less likely to be refracted during fluorescence observation.

As shown in FIG. 5, the four positioning portions 401 are disposed horizontally outside the four chamfered portions 30. The channel member 3 can therefore be easily positioned with respect to the cover member 4 in the horizontal direction.

As shown in FIG. 10, the liquid feed unit 6b includes the elastically deformable balloon pump 63b. The culture medium can therefore be stored. The culture medium can also be pumped using an elastic restoring force without a power supply. That is, it is possible to perform perfusion culture of cells in the culture unit 6c without using a power supply. The balloon pump 63b is contained in the tray 60. This can reduce sagging of the balloon pump 63b due to its own weight when the balloon pump 63b expands (when the balloon pump 63b stores the culture medium therein).

It is herein assumed that, in a method for assembling the MPS device 1, an operator manually combines the cover member 4 and the channel member 3 and then presses the press-in portions 321a of the channel member 3 into the openings 601, 611c, and 612 of the functional units. In this case, the cover member 4 has higher rigidity than the channel member 3. Deformation is therefore less likely to occur. Accordingly, the seven press-in portions 321a of the channel member 3 shown in FIG. 9 need to be pressed into the seven openings 601, 611c, and 612 of the functional units at a time. This work is troublesome.

In this regard, according to the method for assembling the MPS device 1 of the present embodiment, an operator first manually presses the press-in portions 321a of the channel member 3 into the openings 601, 611c, and 612 of the functional units (press-in step). Next, the cover member 4 is attached to the container 2 (fastening step). Therefore, in the press-in step, the operator can press the seven press-in portions 321a into the seven openings 601, 611c, and 612 of the functional units stepwise (e.g., one by one) while bending the flexible channel member 3 as appropriate. This makes the work simple and reliable.

As shown in FIG. 2, the channel member 3 is transparent. The channel member 3 also has rubber elasticity. Therefore, a sample can be collected from the culture unit 6c by inserting, with the cover member 4 removed, an injection needle into the channel member 3 while looking at the culture unit 6c from outside the channel member 3. A drug etc. can also be added to the culture unit 6c. When the injection needle is removed, the puncture hole closes due to the rubber elasticity of the channel member 3.

### Second Embodiment

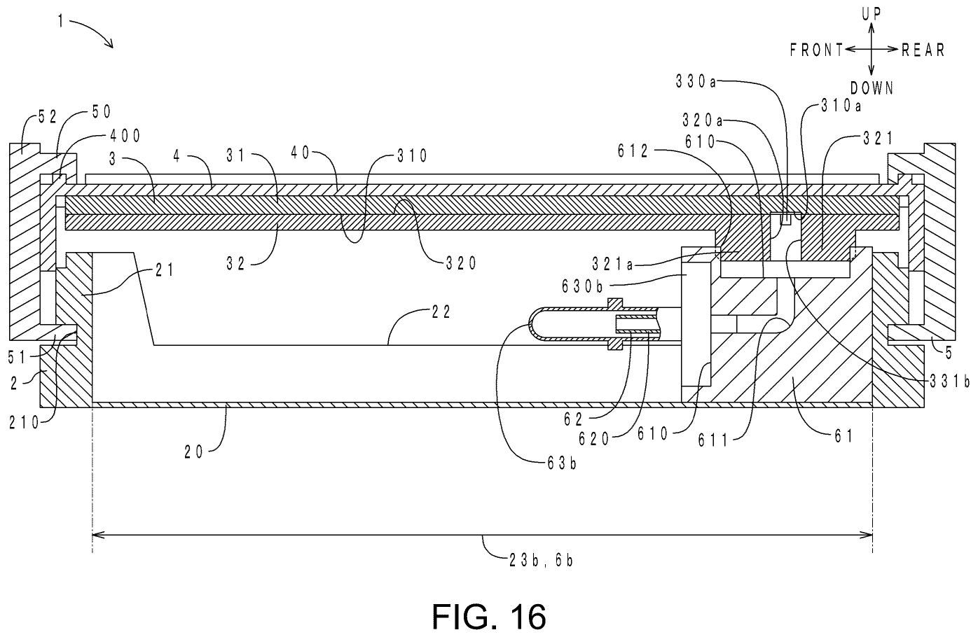

An MPS device of the present embodiment is different from the MPS device of the first embodiment in that no opening is provided in the bottom wall of the container. Another difference is that no tray is placed in the container compartments. Only the differences will be described below. FIG. 16 is a sectional view in the front-rear direction of a portion of the MPS device of the present embodiment in which the liquid feed unit is disposed. The portions corresponding to those in FIG. 12 are denoted by the same signs as those in FIG. 12.

As shown in FIG. 16, the bottom wall 20 does not have the opening 202 (see FIG. 12). The lower surface of the bottom wall 20 are a smooth flat surface. The liquid feed unit 6b docs not include the tray 60 (see FIG. 12). The import member 61 and the balloon pump 63b are directly placed in the container compartment 23b. The import member 61 rests on the bottom wall 20. The balloon pump 63b is supported by the bottom wall 20 when the balloon pump 63b expands.

The MPS device 1 of the present embodiment has the same functions and effects as the MPS device of the first embodiment regarding the portions having the same configurations as the MPS device of the first embodiment. As in the present embodiment, the bottom wall 20 need not necessarily have an opening. Even in this case, sufficient light transmitting properties can be provided by making the bottom wall 20 transparent. As in the present embodiment, the functional units need not necessarily include a tray.

### Third Embodiment

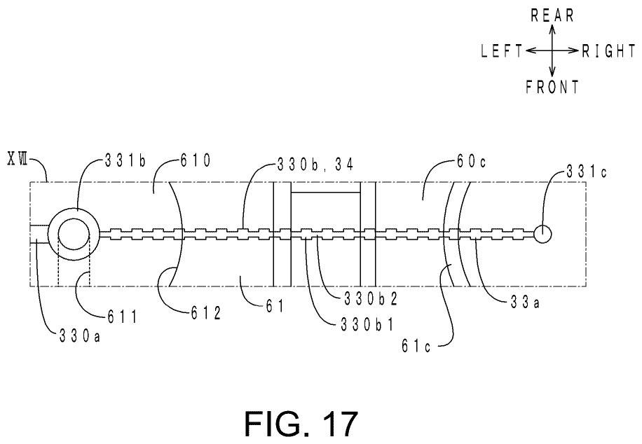

An MPS device of the present embodiment is different from the MPS device of the first embodiment in the channel shape. Only the difference will be described below. FIG. 17 is a transparent partial top view of the MPS device of the present embodiment. The portions corresponding to those in FIG. 3 are denoted by the same signs as those in FIG. 3. FIG. 17 shows a portion corresponding to the region XVII in FIG. 3.

As shown in FIG. 17, the horizontally extending portion 330b of the channel 33a includes a plurality of wide portions 330b1 and a plurality of narrow portions 330b2. The wide portions 330b1 and the narrow portions 330b2 are alternately located in the left-right direction (channel length direction of the horizontally extending portion 330b). The narrow portions 330b2 have a smaller sectional channel area than the wide portions 330b1. Therefore, the sectional channel area of the horizontally extending portion 330b increases and decreases repeatedly along the left-right direction. The horizontally extending portion 330b corresponds to the flow rate control portion 34.

The MPS device of the present embodiment has the same functions and effects as the MPS device of the first embodiment regarding the portions having the same configurations as the MPS device of the first embodiment. As in the present embodiment, the flow rate control portion 34 may adjust the shape in the channel length direction of the channel 33a. The flow rate control portion 34 may adjust the sectional channel shape of the channel 33a. That is, the flow rate control portion 34 need only be configured to adjust at least one of the shape in the channel length direction of the channel 33a and the sectional channel shape of the channel 33a.

### Fourth Embodiment

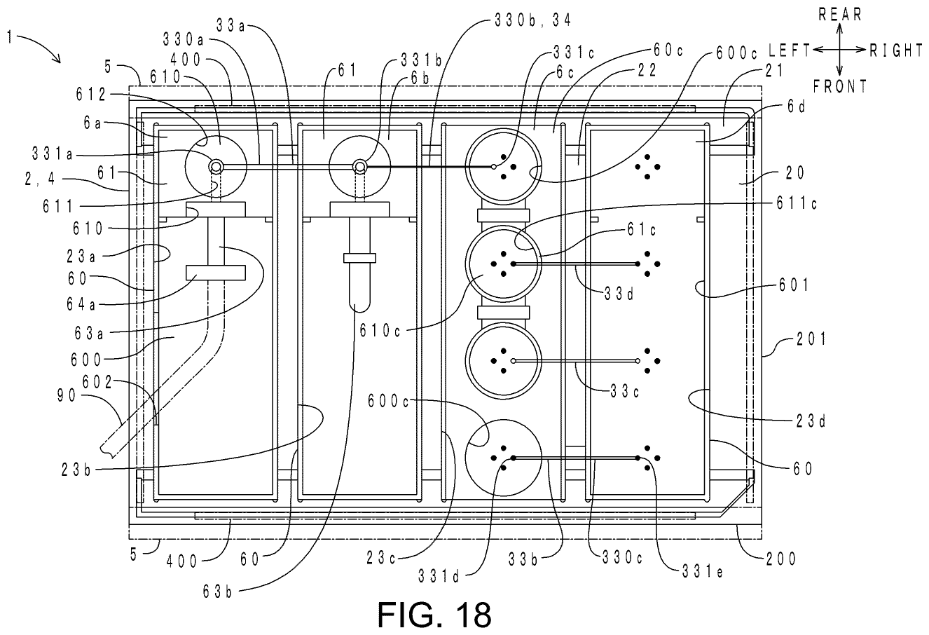

An MPS device of the present embodiment is different from the MPS device of the first embodiment in the channel shape. Only the difference will be described below. FIG. 18 is a transparent top view of the MPS device of the present embodiment. The portions corresponding to those in FIG. 3 are denoted by the same signs as those in FIG. 3.

As shown in FIG. 18, a plurality of channels 33a to 33d is defined in advance in the channel member. When the number and positions of culture vessels 61c to be placed in the culture unit 6c are not yet determined, vertically extending portions of the channels 33a to 33d are scaled. Once the number and arrangement of culture vessels 61c are determined, the vertically extending portion of desired ones of the channels 33a to 33d are opened to open the desired ones of the channels 33a to 33d according to the number and arrangement of culture vessels 61c. In the example shown in FIG. 18, only the channels 33a, 33c are selectively opened out of the channels 33a to 33d according to the number (three) and arrangement of culture vessels 61c. As shown by black circles in the figure, the remaining channels 33b, 33d are sealed.

The MPS device of the present embodiment has the same functions and effects as the MPS device of the first embodiment regarding the portions having the same configurations as the MPS device of the first embodiment. As in this embodiment, the unopened channels 33a to 33d may be formed in advance in the channel member, and only the necessary channels 33a, 33c may be selectively opened after the number of culture vessels 61c to be placed is determined. It should be understood that, this is not limited to the culture vessels 61c of the culture unit 6c, and only necessary ones of the plurality of channels defined in advance in the channel member may be selectively opened according to the number and arrangement of constituent members (tray 60, import member 61, etc.) of any other functional unit.

### Fifth Embodiment

An MPS device of the present embodiment is different from the MPS device of the first embodiment in that there are no cover member, trays, and fastening members. Another difference is that the container compartments of the container extend in the left-right direction (direction in which the long-side portions of the bottom wall of the container extend) and therefore a plurality of culture vessels of the culture unit is connected in the left-right direction. Still another difference is that each of the side and bottom walls of the container has a plurality of openings. Yet another difference is that part (third layer) of the channel member is made of resin. A further difference is that the press-in portion includes an O-ring. The following description mainly focuses on the differences.

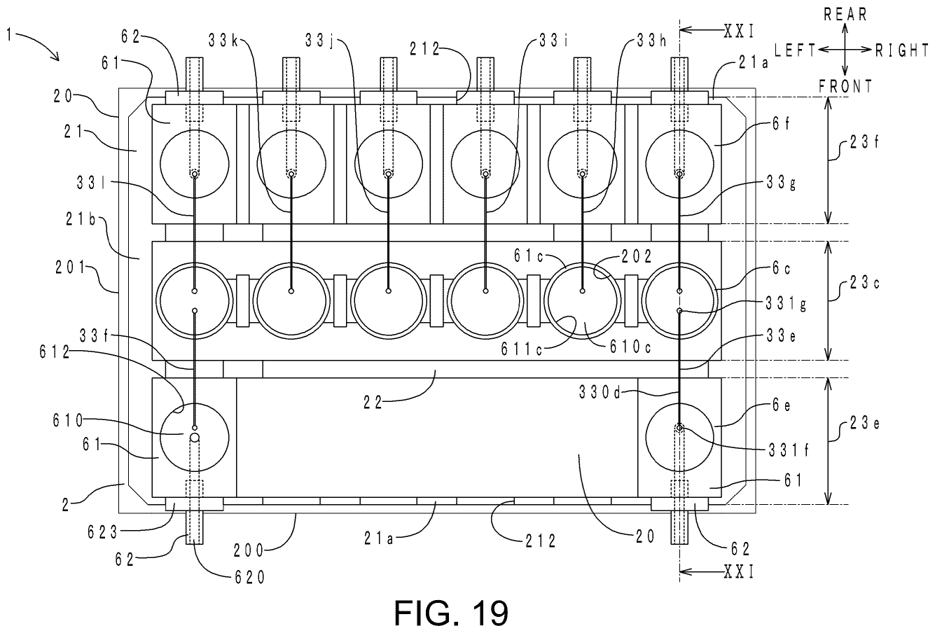

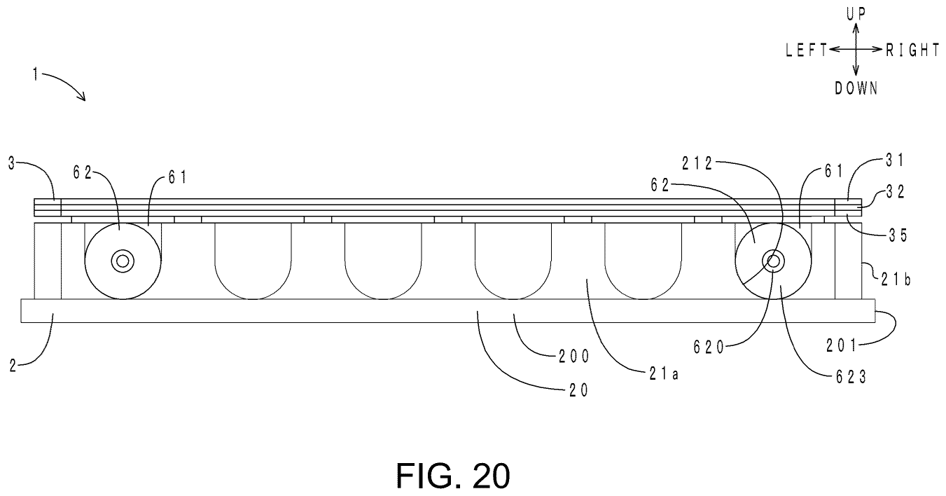

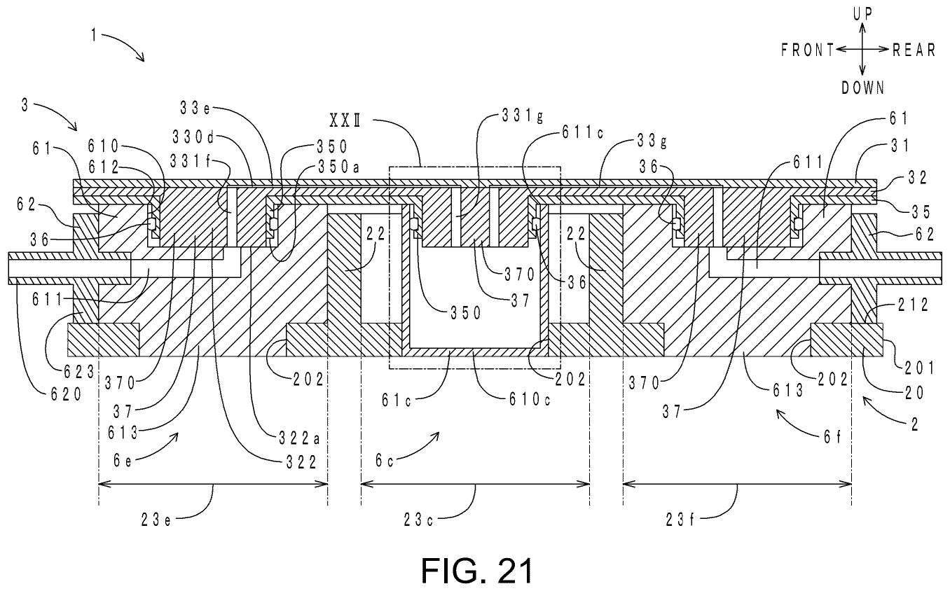

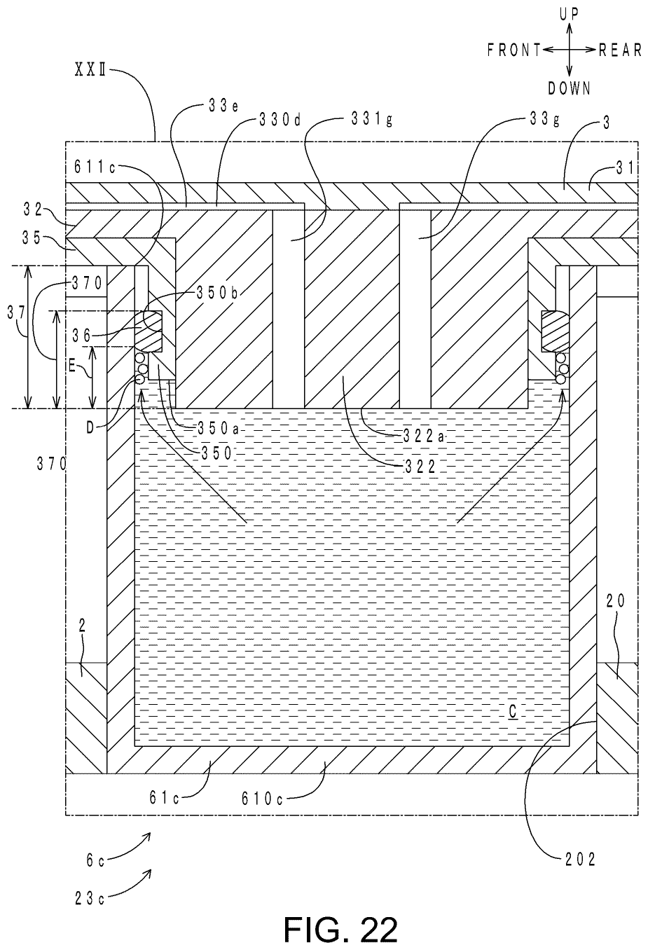

FIG. 19 is a transparent top view of the MPS device of the present embodiment. FIG. 20 is a front view of the MPS device. FIG. 21 is a sectional view in the direction XXI-XXI in FIG. 19. FIG. 22 is an enlarged view of the region XXII in FIG. 21. In these figures, the portions corresponding to those in FIGS. 3, 5, 12, and 13 are denoted by the same signs as those in FIGS. 3, 5, 12, and 13.

### [Configuration of MPS Device 1]

First, the configuration of the MPS device of the present embodiment will be described. As shown in FIGS. 19 to 22, the MPS device 1 includes the container 2, the channel member 3, a first connecting unit 6e, the culture unit 6c, and a second connecting unit 6f. The first connecting unit 6e, the culture unit 6c, and the second connecting unit 6f are included in the concept of the “functional unit” in the present disclosure.

### (Container 2)

As shown in FIGS. 19 to 21, the container 2 includes the bottom wall 20, four side walls 21a, 21b, two partition walls 22, and container compartments 23e, 23c, and 23f. The bottom wall 20 is in the form of a rectangular plate extending in the horizontal direction. The bottom wall 20 has 14 openings 202 (same number as the total number of import members 61 and culture vessels 61c).

Of the four side walls 21a, 21b, the pair of front and rear side walls 21a stands upward from the pair of front and rear long-side portions 200. The side walls 21a extend in the left-right direction (direction in which the long-side portions 200 extend). The pair of left and right side walls 21b stands upward from the pair of left and right short-side portions 201. The side walls 21b extend in the front-rear direction (direction in which the short-side portions 201 extend). As shown in FIG. 20, each side wall 21a has six openings 212. The six openings 212 are located next to each other in the left-right direction. The opening 212 has a U-shape that is recessed downward.

As shown in FIG. 19, the two partition walls 22 connect the pair of left and right side walls 21b to each other. The two partition walls 22 are located next to each other in the front-rear direction and parallel to the pair of side walls 21a. The partition walls 22 extend in the left-right direction. The two partition walls 22 divide the space inside the container 2 into the three container compartments 23c, 23c, and 23f.

The three container compartments 23e, 23c, and 23f have the same size as viewed in the up-down direction. The container compartments 23c, 23c, and 23f are compatible with the plurality of functional units. As shown in FIG. 19, the container compartments 23c, 23c, and 23f extend in the left-right direction. The three container compartments 23c, 23c, and 23f are located next to each other in the front-rear direction with the two partition walls 22 interposed therebetween. Of these container compartments, each of the bottom surfaces of the container compartments 23c, 23f (upper surface of the bottom wall 20) has six openings 202 located next to each other in the left-right direction. The container compartment 23e has two openings 202 located spaced apart in the left-right direction.

### (Channel Member 3)

As shown in FIGS. 21 and 22, the channel member 3 fixes the functional units (first connecting unit 6e, culture unit 6c, and second connecting unit 6f). The channel member 3 seal the openings 611c, 612 of the functional units (specifically, openings 611c of six culture vessels 61c of the culture unit 6c, upper openings 612 of two import members 61 of the first connecting unit 6e, and upper openings 612 of six import members 61 of the second connecting unit 6f) in a liquid-tight manner.

As shown in FIGS. 19, 21, and 22, the channel member 3 includes the first layer 31 made of PDMS, the second layer (soft layer) 32 made of PDMS, a third layer (hard layer) 35 made of resin, eight channels 33e to 331, and 14 contact portions 37 (same number as the total number of import members 61 and culture vessels 61c). The first layer 31, the second layer 32, and the third layer 35 are stacked in this order from top to bottom. The third layer 35 has higher rigidity than the first layer 31 and the second layer 32.

As shown in FIG. 22, the second layer 32 includes 14 second-layer protrusions (soft-layer protrusions) 322 protruding downward. The second-layer protrusions 322 have a short cylindrical shape. The third layer 35 includes 14 third-layer protrusions (hard-layer protrusions) 350 protruding downward. The third-layer protrusions 350 have a short hollow cylindrical shape. The outer peripheral surface of each third-layer protrusion 350 has an annular groove 350b. An O-ring (seal member) 36 made of an elastomer is disposed in the annular groove 350b. The O-ring 36 has rubber elasticity. The second-layer protrusion 322 is disposed radially inside the third-layer protrusion 350. The second-layer protrusion 322 extends through the third-layer protrusion 350 in the up-down direction. The third-layer protrusion 350 has higher rigidity than the second-layer protrusion 322 and the O-ring 36.

As shown in FIGS. 21 and 22, the contact portions 37 are located under the third layer 35 (plate portion other than the third-layer protrusions 350). The contact portions 37 are disposed so as to face the functional units. In the front container compartment 23c, the contact portions 37 contact the first connecting unit 6e (recesses 610 of the import members 61) from above. In the rear container compartment 23f, the contact portions 37 contact the second connecting unit 6f (recesses 610 of the import members 61) from above. In the middle container compartment 23c in the front-rear direction, the contact portions 37 are inserted in the culture unit 6c (openings 611c of the culture vessels 61c) from above.

As shown in FIGS. 21 and 22, each contact portion 37 includes the second-layer protrusion 322, the third-layer protrusion 350, and the O-ring 36. The O-ring 36 and a portion of the contact portion 37 that is located below the O-ring 36 are a press-in portion 370. The press-in portions 370 are pressed into the openings 611c, 612 of the functional units. Each press-in portion 370 includes the second-layer protrusion 322 made of PDMS, the third-layer protrusion 350 made of resin, and the O-ring 36 made of an elastomer. That is, the press-in portions 370 are made of a material containing PDMS and resin. The O-rings 36 of the press-in portions 370 elastically contact the inner peripheral surfaces of the openings 611c, 612 from radially inside the openings 611c, 612. This clastic contact provides liquid-tight scaling of the openings 611c, 612.

As shown in FIG. 22, when looking at the positional relationship in the up-down direction among the O-ring 36, a lower surface (back-side protrusion end) 350a of the third-layer protrusion 350, and a lower surface (back-side protrusion end) 322a of the second-layer protrusion 322 in the press-in portion 370, these members and portions are located from top to bottom in the order of the O-ring 36, the lower surface 350a of the third-layer protrusion 350, and the lower surface 322a of the second-layer protrusion 322. When looking at the positional relationship in the radial direction (radial direction about the central axis of the cylindrical shape of the second-layer protrusion 322) among the O-ring 36, the lower surface 350a of the third-layer protrusion 350, and the lower surface 322a of the second-layer protrusion 322 in the press-in portion 370, these members and portions are located from outside to inside in the radial direction in the order of the O-ring 36, the lower surface 350a of the third-layer protrusion 350, and the lower surface 322a of the second-layer protrusion 322. As described above, the press-in portions 370 (lower end portions of the contact portions 37) have a stepped shape (stepped tapered shape) that becomes thinner toward its bottom.

As shown in FIG. 19, the eight channels 33e to 331 extend across adjacent two of the container compartments 23c, 23c, and 23f. Specifically, the channels 33c, 33f extend across the container compartments 23c, 23c, and the channels 33g to 331 extend across the container compartments 23c, 23f. The eight channels 33e to 331 have the same configuration. The eight channels 33c to 331 have the same configuration as the channel 33b shown in FIGS. 3 and 6. The configuration of the channel 33e at the right front end will be described below as a representative of the eight channels 33e to 331.

As shown in FIGS. 21 and 22, the channel 33e is composed of a horizontally extending portion 330d and a pair of front and rear vertically extending portions 331f, 331g. The horizontally extending portion 330d is formed at the boundary between the first layer 31 and the second layer 32. The horizontally extending portion 330d extends in the front-rear direction (horizontal direction). The vertically extending portions 331f, 331g are located in the second layer 32. The vertically extending portions 331f, 331g extend in the up-down direction. The upper ends of the vertically extending portions 331f, 331g are continuous with the horizontally extending portion 330d. The lower ends of the vertically extending portions 331f, 331g are open to the lower surface of the contact portion 37 (press-in portion 370). The vertically extending portion 331f is open to the first connecting unit 6e in the container compartment 23e, and the vertically extending portion 331g is open to the culture unit 6c in the container compartment 23c.

### (Functional Units)

As shown in FIG. 19, the container 2 is divided into the three container compartments 23c, 23c, and 23f located next to each other in the front-rear direction. The first connecting unit 6e is detachably placed in the front container compartment 23e, the culture unit 6c is detachably placed in the middle container compartment 23c in the front-rear direction, and the second connecting unit 6f is detachably placed in the rear container compartment 23f.

### (First Connecting Unit 6e)

The first connecting unit 6e is disposed in the container compartment 23e. The first connecting unit 6e connects the inside and outside of the MPS device 1. That is, the culture medium is supplied from outside to inside (e.g., the culture unit 6c etc.) via the first connecting unit 6e. Waste liquid is discharged from inside to outside (e.g., a waste liquid tank etc.) via the first connecting unit 6c.

The first connecting unit 6e includes two import members 61. The import members 61 are disposed at both ends in the left-right direction of the first connecting unit 6e. The two import members 61 have the same configuration. The configuration of the right import member 61 will be described below as a representative of the two import members 61.

The import member 61 is made of resin and has the shape of a rectangular parallelepiped. As shown in FIG. 21, the import member 61 has a recess 610 in its upper surface. The recess 610 in the upper surface communicates with the front surface via the communication hole 611 extending in an L-shape.

The press-in portion 370 of the contact portion 37 of the channel member 3 is pressed into the opening 612 of the recess 610 in the upper surface. The communication hole 611 of the import member 61 is connected to the channel 33e (vertically extending portion 331f) via the press-in portion 370. The connector 62 is detachably mounted on the front surface of the import member 61. The connector 62 includes the tube member 620 and a flange 623. The flange 623 is fitted on the tube member 620. As shown in FIGS. 19 to 21, the flange 623 is placed in the opening 212 of the side wall 21a (rightmost opening 212 of the container compartment 23c). As shown in FIG. 21, a portion of the tube member 620 that is located rearward (inward) of the flange 623 is inserted in the communication hole 611. A protrusion 613 protrudes from the lower surface of the import member 61. The protrusion 613 is fitted in the opening 202 of the bottom wall 20 (rightmost opening 202 of the container compartment 23e). A tube, an infusion pump (such as a syringe pump), a waste liquid tank, a check valve, etc. can be connected to the connector 62.

### (Culture Unit 6c)

As shown in FIGS. 21 and 22, the culture unit 6c is disposed in the container compartment 23c. The culture unit 6c is connected to the first connecting unit 6e via the channel 33e. The culture unit 6c is connected to the second connecting unit of via the channel 33g. The culture medium is supplied from at least one of the first connecting unit 6e and the second connecting unit of to the culture unit 6c. Waste liquid is discharged from the culture unit 6c to at least one of the first connecting unit 6e and the second connecting unit 6f.

The configuration of the culture unit 6c of the present embodiment (FIGS. 19, 21, and 22) is generally the same as the configuration of the culture unit 6c shown in FIGS. 10 and 13. The difference is that the culture unit 6c includes six culture vessels 61c connected in the left-right direction (direction in which the container 2 (long-side portions 200 of the bottom wall 20) extends). Another difference is that the culture unit 6c does not include the base 60c shown in FIGS. 10 and 13. Still another difference is that the bottom walls 610c of the culture vessels 61c are fitted in the openings 202 of the bottom wall 20.

As shown in FIG. 22, the press-in portion 370 of the contact portion 37 of the channel member 3 is pressed into the opening 611c of the culture vessel 61c. The culture vessel 61c is connected to the channels 33e (vertically extending portion 331g), 33g via the press-in portion 370.

### (Second Connecting Unit 6f)

As shown in FIG. 21, the second connecting unit 6f is disposed in the container compartment 23f. The second connecting unit of connects the inside and outside of the MPS device 1. The configuration of the second connecting unit 6f is generally the same as the configuration of the first connecting unit 6e. The difference is that the second connecting unit 6f includes six import members 61 located next to each other in the left-right direction. The arrangement of the import members 61 of the second connecting unit 6f is symmetrical with the arrangement of the import members 61 of the first connecting unit 6c. The import members 61 of the second connecting unit 6f have the same configuration as the import members 61 of the first connecting unit 6c.

### [Functions and Effects]

Next, functions and effects of the MPS device of the present embodiment will be described. The MPS device of the present embodiment has the same functions and effects as the MPS device of the first embodiment regarding the portions having the same configurations as the MPS device of the first embodiment.

As shown in FIGS. 20 and 21, each side wall 21a has openings 212. The openings 212 extend through the side walls 21a in the front-rear direction (inside-outside direction of the container 2). Therefore, the outside of the container 2 and the inside of the container 2 can be easily connected through the openings 212. Specifically, the connectors 62 of the import members 61 extend through the side walls 21a through the openings 212. The outside of the container 2 and the inside of the container 2 can be easily connected by the connectors 62.

As shown in FIGS. 19 and 21, the bottom wall 20 has the plurality of openings 202. The openings 202 facilitate positioning of the functional units. Specifically, the protrusions 613 of the import members 61 of the first connecting unit 6e are fitted in the openings 202. The first connecting unit 6e can therefore be easily positioned. Similarly, the protrusions 613 of the import members 61 of the second connecting unit 6f are fitted in the openings 202. The second connecting unit 6f can therefore be easily positioned. Similarly, the culture vessels 61c of the culture unit 6c are fitted in the openings 202. The culture unit 6c can therefore be easily positioned.

As shown in FIGS. 21 and 22, the press-in portions 370 of the contact portions 37 of the channel member 3 are pressed into the openings 612, 611c of the functional units (first connecting unit 6e, culture unit 6c, and second connecting unit 6f). The functional units and the container 2 can therefore be easily positioned with respect to the channel member 3.

Specifically, regarding positioning in the horizontal direction, the central axis of each press-in portion 370 (axis passing through the centroid (center of gravity of the geometrical figure) of a horizontal section of the press-in portion 370 and extending in the up-down direction) can be made to coincide (align) with the central axis of the corresponding opening 612, 611c (axis passing through the centroid (center of gravity of the geometrical figure) of a horizontal section of the opening 612, 611c and extending in the up-down direction) by pressing the press-in portion 370 into the corresponding opening 612, 611c. The functional units and the container 2 can thus be easily positioned with respect to the channel member 3 in the horizontal direction.

Regarding positioning in the up-down direction (vertical direction), the lower surface of the third layer 35 (portions surrounding the roots of the third-layer protrusions 350) can be brought into contact with the edges of the openings 611c of the culture vessels 61c by pressing the press-in portions 370 into the openings 612, 611c. The third layer 35 and the culture vessels 61c are both made of resin. The third layer 35 and the culture vessels 61c therefore have higher rigidity compared to the case where these members are made of an elastomer. The amount by which the press-in portions 370 are pressed into the openings 612, 611c can thus be determined. Accordingly, the functional units and the container 2 can be easily positioned with respect to the channel member 3 in the up-down direction.

As described above, according to the MPS device 1 of the present embodiment, the functional units and the container 2 can be easily positioned with respect to the channel member 3 by merely pressing the press-in portions 370 into the openings 612, 611c. Moreover, the channel member 3 can be easily attached to the functional units and container 2. Therefore, the cover member 4 and the fastening members 5 shown in FIG. 13 are not necessary.

Pressing the press-in portions 370 into the openings 612, 611c can improve sealing between the channel member 3 and the openings 612, 611c. Specifically, the press-in portion 370 shown in FIG. 22 includes the second-layer protrusion 322 made of PDMS, the third-layer protrusion 350 made of resin, and the O-ring 36 made of an elastomer. The third-layer protrusion 350 has higher rigidity than the O-ring 36. The side wall of the culture vessel 61c has higher rigidity than the O-ring 36. Therefore, when the press-in portion 370 is pressed into the opening 611c, the O-ring 36 is preferentially elastically deformed. The elastic contact force (elastic restoring force) of the O-ring 36 provides sealing between the outer peripheral surface of the third-layer protrusion 350 and the inner peripheral surface of the opening 611c (side wall) of the culture vessel 61c. The press-in portions 370 of the first connecting unit 6e and second connecting unit 6f have the same effects as those of the press-in portions 370 of the culture unit 6c described above.

The clastic contact force of the O-ring 36 of the press-in portion 370 shown in FIG. 22 acts on the third-layer protrusion 350 in a direction in which the third-layer protrusion 350 is reduced in diameter (direction in which the third-layer protrusion 350 is compressed). The third-layer protrusion 350 has higher rigidity than the second-layer protrusion 322. Therefore, the clastic contact force (compressive force) of the O-ring 36 is less likely to be transmitted to the second-layer protrusion 322 via the third-layer protrusion 350. This can reduce deformation of the channels 33c (vertically extending portion 331g), 33g inside the second-layer protrusion 322 due to the clastic contact force of the O-ring 36. The press-in portions 370 of the first connecting unit 6c and second connecting unit 6f have the same effects as those of the press-in portions 370 of the culture unit 6c described above.

It is herein assumed that each third-layer protrusion 350 shown in FIG. 22 is made of a flexible elastomer, that is, the entire press-in portion 370 is made of a flexible elastomer (this case is also included in the concept of the microphysiological system device of the present disclosure). In this case, when pressing the press-in portions 370 into the openings 612, 611c, part of the downward pressing force applied from the channel member 3 to the functional units is likely to be consumed by deformation of the flexible press-in portions 370. In other words, the pressing force tends to escape. Accordingly, depending on the type and arrangement of the functional units, it may be difficult to perform the work of pressing the press-in portions 370 into the openings 612, 611c.

In this regard, each third-layer protrusion 350 shown in FIG. 22 is made of resin having high rigidity. Therefore, when pressing the press-in portions 370 into the openings 612, 611c, part of the downward pressing force applied from the channel member 3 to the functional units is less likely to be consumed by deformation of the press-in portions 370. In other words, the pressing force is less likely to escape. Accordingly, depending on the type and arrangement of the functional units, it may become easier to perform the work of pressing the press-in portions 370 into the openings 612, 611c.

During microscopic observation, the inside of the culture vessel 61c shown in FIG. 22 is observed from below with an inverted fluorescence microscope while being irradiated with light from above. Therefore, it is preferable from the viewpoint of visibility that the culture vessel 61c be filled with the culture medium C. Actually, however, gas bubbles D may enter the culture vessel 61c and may interfere with microscopic observation.

In this regard, as described above, the press-in portions 370 (lower ends of the contact portions 37) have a stepped shape (stepped tapered shape) that becomes thinner toward their bottom (in the direction in which gravity acts). This allows the gas bubbles D having entered the culture vessel 61c to move upward and into a gas bubble storage space (gas bubble storage portion) E (specifically, an annular space located immediately under the O-ring 36 and defined in the clearance between the inner peripheral surface of the side wall of the culture vessel 61c located on the radially outer side and the outer peripheral surface of the third-layer protrusion 350 located on the radially inner side (or between the outer peripheral surface of the third-layer protrusion 350 and the outer peripheral surface of the second-layer protrusion 322)) due to the buoyancy of the gas bubbles D themselves. Therefore, the gas bubbles D are less likely to interfere with microscope observation. This improves visibility during microscopic observation.

As shown in FIG. 22, the bottom wall 610c of each culture vessel 61c is exposed from the lower side of the container 2 through the opening 202 in the bottom wall 20 of the container 2. The lower surface of the bottom wall 610c is flush with the lower surface of the bottom wall 20. This makes it easier to adjust the focal length of an inverted fluorescence microscope compared to the case where there is no opening 202 (case where the bottom wall 20 is solid and the culture vessel 61c rests on the upper surface of the bottom wall 20). The lower surface 322a of the second-layer protrusion 322 (lower end face of the contact portion 37) has an angular outer edge. That is, corners are disposed at the outer edge. The outer edge has no chamfered portion.

This improves visibility during microscopic observation.

### <Others>

The embodiments of the microphysiological system device of the present disclosure have been described above. However, embodiments are not particularly limited to those described above. The present disclosure can be carried out in various modified or improved forms that can be made by those skilled in the art.

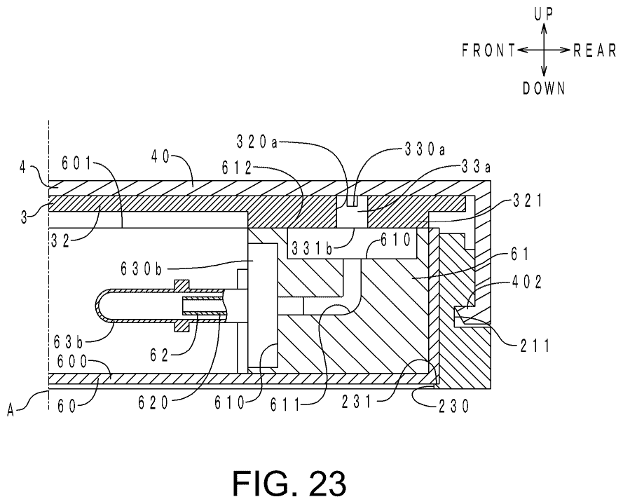

FIG. 23 is a partial sectional view in the front-rear direction of an MPS device of a further embodiment. The portions corresponding to those in FIG. 15 are denoted by the same signs as those in FIG. 15. As shown in FIG. 23, the cover member 4 may include an engaging claw 402. The container 2 may have an engaged recess 211. The cover member 4 and the container 2 may be combined by engaging the engaging claw 402 with the engaged recess 211. The channel member 3 may have a single-layer structure. The channel 33a may be formed at the boundary between the cover member 4 and the channel member 3. The contact portion 321 may be in contact (including elastic contact) with the edge of the opening 612 of the import member 61 along the entire circumference. That is, the contact portion 321 need not necessarily include the press-in portion 321a (see FIG. 12).

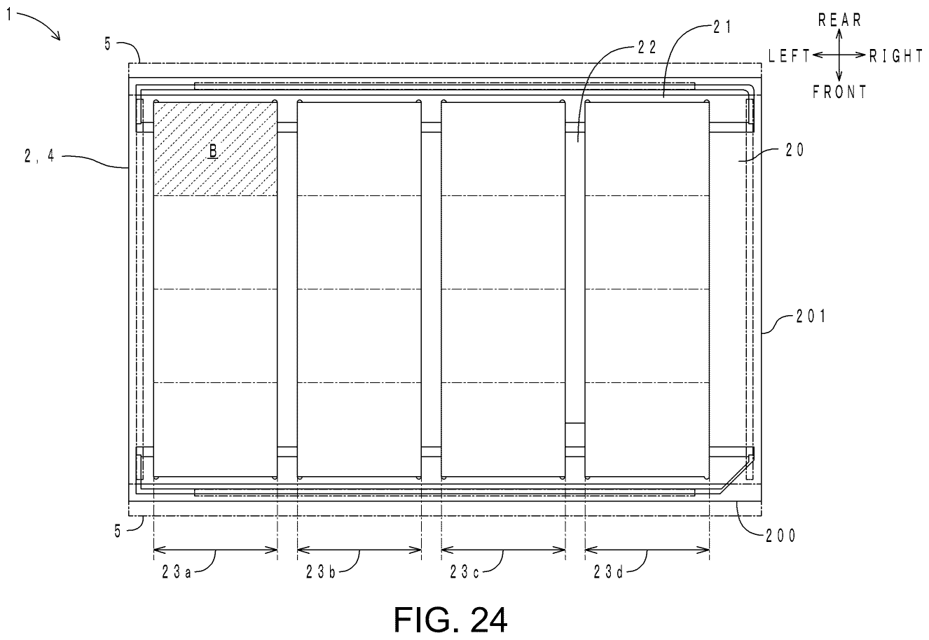

FIG. 24 is a schematic top view of the MPS device of the present disclosure. The portions corresponding to those in FIG. 3 are denoted by the same signs as those in FIG. 3. As shown in FIG. 24, each of the container compartments 23a to 23d can be divided into four blocks (hatched area) B connected in the front-rear direction. The sizes of the constituent members of the functional units (import members 61, balloon pump 63b, and trays 60 shown in FIG. 3, import members 61 shown in FIG. 19, etc.) are preferably integer multiples of the size of the block B, as viewed in the front-back direction. For example, it is preferable that the import members 61 have a size equivalent to one block B, the balloon pump 63b have a size equivalent to three blocks B, and the trays 60 have a size equivalent to four blocks B. Determining the sizes of the constituent members based on the size of the block B in this manner improves space efficiency of the container compartments 23a to 23d. This also facilitates rearrangement of the constituent members of the functional units.

The constituent members of the MPS device 1 according to the plurality of embodiments shown in FIGS. 1 to 24 can be rearranged as appropriate. For example, the engaging claw 402 and the engaged recess 211 shown in FIG. 23 may be applied to the MPS devices 1 shown in FIGS. 12 and 16. The channel member 3 having a single-layer structure shown in FIG. 23 may be applied to the MPS devices 1 shown in FIGS. 12, 16, and 21. The channel member 3 having a two-layer structure shown in FIGS. 12 and 16 may be applied to the MPS devices 1 shown in FIGS. 21 and 23. The channel member 3 having a three-layer structure shown in FIG. 21 may be applied to the MPS devices 1 shown in FIGS. 12, 16, and 23. The flow rate control portion 34 shown in FIG. 17 may be applied to the MPS devices 1 shown in FIGS. 21 and 23.

Regarding the number of functional units (supply unit 6a, liquid feed unit 6b, culture unit 6c, waste liquid storage unit 6d, first connecting unit 6e, and second connecting unit 6f), the number of functional units that are placed in a single container 2 is not particularly limited. It may be one, or may be two or more. The arrangement of the functional units in a single container 2 is not particularly limited. The number of functional units that are placed in a single container compartment 23a to 23f is not particularly limited. It may be one, or may be two or more. The arrangement of the functional units in a single container compartment 23a to 23f is not particularly limited. The number of constituent members of the functional unit is not particularly limited. It may be one, or may be two or more.

In the supply unit 6a shown in FIG. 10, a member (e.g., a tank for storing the culture medium) may be disposed in front of the import member 61. A different valve (such as a three-way valve) may be disposed instead of the check valve 64a. The connection mechanism between the tube 63a and the import member 61 is not particularly limited. The type of pump for the liquid feed unit 6b is not particularly limited. It may be a micropump etc. The connection mechanism between the pump and the import member 61 is not particularly limited.

The number of culture vessels 61c placed (connected) in the culture unit 6c is not particularly limited. It may be one, or may be two or more. The material of the filters 62c is not particularly limited. Any material may be used as long as it selectively allows a desired substance to pass therethrough. The waste liquid storage unit 6d may be the tray 60 or a bag. The use of the culture unit 6c is not particularly limited. The culture unit 6c may be used to culture bacteria, yeast, algae, etc. in addition to cells.

A pump unit for circulating the culture medium in the culture unit 6c may be disposed instead of the waste liquid storage unit 6d. The culture medium can thus be continuously supplied to the culture unit 6c. This improves biomimetic properties (reproduction of the in vivo environment) of the MPS device 1.

The types of functional units are not particularly limited. For example, the functional units may be a stirring unit including a stirrer for stirring the culture medium, a sensor unit for detecting a desired signal (electrical signal, optical signal, etc.) from the culture unit 6c, a stress applying unit for applying desired stress (vibration, pressure, etc.) to cells in the culture unit 6c, etc. A plurality of functional units of the same type may be disposed in a plurality of container compartments 23a to 23d. For example, the liquid feed unit 6b may be disposed in each of the container compartment 23b and the container compartment 23d shown in FIG. 3. The culture medium and a drug may be supplied from the liquid feed unit 6b in the container compartment 23b and the liquid feed unit 6b in the container compartment 23d to the culture unit 6c, respectively. Various types of external equipment may be connected to the MPS device 1. Examples of the external equipment include an external pump (may be a pump other than the balloon pump 63b), a large waste liquid tank, and a sensor.

The shapes, sizes, positions, numbers, materials, and transparencies (hereinafter collectively referred to as “shapes etc.”) of the cover member 4, channel member 3, container 2, fastening members 5, and functional units are not particularly limited. The degree of transparency of the cover member 4, channel member 3, and container 2 is not limited. They may be colorless and transparent, or may be colored and transparent. They may have any degree of transparency as long as at least the portion corresponding to the culture unit 6c is transparent as viewed in the front-back direction. Depending on the object to be analyzed, the portion corresponding to the culture unit 6c may be opaque. It is preferable that the materials of these members are ones that neither deform nor deteriorate in the environment inside the incubator. The materials of the container 2, fastening members 5, and trays 60 are not particularly limited. They may be polycarbonate, acrylic, polystyrene, etc. The material of the import members 61 is not particularly limited. It may be polystyrene, PDMS, etc.

As shown in FIGS. 10, 12 and 21, the container 2 may have the opening 202 in the bottom wall 20. This makes it easier to observe the portion to be observed (culture unit 6c). As shown in FIG. 16, the bottom wall 20 need not necessarily have an opening. This facilitates scaling (airtightness, liquid-tightness, etc.) of the container 2 (at least one of the container compartments 23a to 23d). For example, this allows cells etc. to be cultured under internal pressure. When the bottom wall 20 does not have an opening, the thickness of the bottom wall 20 is preferably 1 mm or less. This configuration is less likely to interfere with observation of the portion to be observed. The use of the MPS device 1 is not particularly limited. For example, it can be used for observing cells, observing biological signals, and producing new substances.

The shape of the openings 212 of the side walls 21a shown in FIG. 20 is not particularly limited. For example, they may be endless holes (perfect circular shape, elliptical shape, or polygonal shape (triangle, quadrilateral, pentagon, hexagon, etc.)), or may be cuts with ends (U-shape, C-shape, V-shape, polygon, etc.). The openings 212 may have any shape as long as they extend through the side walls 21a. The openings 212 may be located in the side walls 21b.

The material of the channel member 3 is not particularly limited. It may be an elastomer, or may be resin. In the case of resin, polystyrene, COP (cycloolefin polymer), COC (cycloolefin copolymer), etc. may be used. The entire channel member 3 may be made of an elastomer. The entire channel member 3 may be made of resin. Alternatively, part of the channel member 3 may be made of an elastomer, and the remaining part may be made of resin. For example, when the channel member 3 has a single-layer structure, part of the channel member 3 may be made of an elastomer and the remaining part may be made of resin. When the channel member 3 has a multi-layer structure, any desired layer (single layer or two or more layers) may be made of an elastomer and the remainder may be made of resin. The channel member 3 may or may not have elasticity (including rubber elasticity). The elasticity of the channel member 3 may be provided by a different member (gasket, O-ring (e.g., the O-ring 36 shown in FIG. 22), etc.). For example, a gasket may be interposed between the lower surface of the contact portion 321 and the upper surface of the import member 61 (around the recess 610) shown in FIG. 23.

The material of the O-ring (seal member) 36 shown in FIG. 22 is not particularly limited. It may be made of an elastomer or resin. The O-ring 36 may be made of any material as long as the clastic contact force of the O-ring 36 provides sealing between the outer peripheral surface of the third-layer protrusion 350 and the inner peripheral surface of the opening 611c of the culture vessel 61c.

The number of layers of the channel member 3 is not particularly limited. For example, a third layer may be stacked on the first layer 31 and the second layer 32, and the horizontally extending portions 330a to 330d may be defined at each of the boundary between the first layer 31 and the second layer 32 and the boundary between the second layer 32 and the third layer. This allows the horizontally extending portions 330a to 330d to cross each other in a three-dimensional manner as viewed in the front-back direction.

The direction in which the channels 33a to 331 extend is not particularly limited. This direction may be the front-back direction, the layer direction, or a direction crossing the front-back direction and the layer direction. The channels 33a to 331 may have a linear shape, a curved shape, a combination of linear and curved shapes, etc. in the channel length direction. The shapes (sectional channel shapes) of the channels 33a to 331 in a direction perpendicular to the channel length direction may be a polygon (triangle, quadrilateral, hexagon, etc.), a circle (perfect circle, ellipse), a semicircle, etc. The channels 33a to 331 (specifically, at least part of at least one of the channels 33a to 33l) may form a so-called Tesla valve. This can reduce backflow of the fluid without a movable part such as a check valve.

The method for defining the channels 33a to 33d is not particularly limited. When the MPS device 1 includes the cover member 4, the channels 33a to 33d may be defined at the boundary between the cover member 4 and the channel member 3. The channels 33a to 331 may have branches. The channels 33a to 331 where the flow rate control portion 34 is disposed (whose flow rate is to be controlled) are not particularly limited. The flow rate control portion 34 may be disposed in at least one of the channels 33a to 331. Either or both of the cover member 4 and the fastening members 5 may be omitted.

The use of the channels 33a to 331 is not particularly limited. For example, the channels 33a to 331 (specifically, at least one of the channels 33a to 33l) may be used for degassing the functional unit. Specifically, the channels 33a to 33d shown in FIG. 18 may be used for degassing the culture unit 6c. In this case, gas bubbles may be stored in the channels 33a to 33d themselves. That is, the channels 33a to 33d themselves may serve as gas bubble storage portions. A gas bubble storage space E shown in FIG. 22 can thus be provided in the channels 33a to 33d themselves. The channels 33a to 33d may be caused to communicate with a gas bubble storage portion that is a separate member from the channels 33a to 33d. The gas bubble storage space E shown in FIG. 22 can thus be provided in the member other than the channels 33a to 33d. The effects of the gas bubble storage space E in the fifth embodiment described above (FIG. 22) can be obtained by disposing the gas bubble storage portion in this manner.

It is herein assumed that the MPS device 1 including the culture unit 6c is used in an environment where a sudden pressure drop may occur (e.g., outer space etc.). In this case, the pressure drop may lead to larger gas bubbles in the culture medium (gas may be air or a gas other than air), which may result in the culture unit 6c not working properly.

In this regard, causing the channels 33a to 33d to communicate with the gas bubble storage portion allows the culture vessels 61c of the culture unit 6c to be degassed via the channels 33a to 33d. This can reduce the possibility of the culture unit 6c not working properly due to gas bubbles. The same applies to the channels 33c to 331 shown in FIG. 19.

The gas bubble storage portion may be disposed inside the container 2 (inside the MPS device 1). Gas bubble management can thus be completed inside the MPS device 1. This improves handleability compared to the case where the gas bubble storage portion is disposed outside the container 2.

The gas bubble storage portion may be disposed outside the container 2 (outside the MPS device 1). This increases design flexibility in size, shape, etc. of the gas bubble storage portion compared to the case where the gas bubble storage portion is disposed inside the container 2. This also reduces the impact of any trouble caused by gas bubbles on the MPS device 1 compared to the case where the gas bubble storage portion is disposed inside the container 2.

The shapes etc. of the contact portions 321, 37 are not particularly limited. The numbers of contact portions 321, 37 may be either the same as or different from the number of functional units that need to be fixed. The shapes etc. of the press-in portions 321a, 370 are not particularly limited. The numbers of press-in portions 321a, 370 may be either the same as or different from the number of functional units having the openings 601, 611c, and 612. Regarding the shapes of the press-in portions 321a, 370, the taper angle is not limited. The tapered shape may have either a planar tapered shape or a curved tapered shape. The press-in portions 321a, 370 need not necessarily have a tapered shape. The press-in portions 321a, 370 may have a sloped or stepped shape that becomes thinner toward their bottom. The press-in portions 321a, 370 may have any shape as long as their sectional areas (more specifically, sectional areas in a direction (horizontal direction) perpendicular to the axial direction (up-down direction) of the press-in portions 321a, 370) become smaller toward their bottom. It should be understood that the press-in portions 321a, 370 may have a constant sectional area along their entire length in the up-down direction. The number of openings of the channels 33a to 331 for a single contact portion 321, 37 is not particularly limited. It may be one, or may be two or more. The direction in which the MPS device 1 is installed is not particularly limited. The central axis A shown in FIG. 12 may extend in the vertical direction, the horizontal direction, or a direction crossing the vertical and horizontal directions.

1. A microphysiological system device comprising:
a container in which a plurality of functional units is placed in a replaceable manner; and
a channel member disposed on a front side of the container and defining at least part of a channel that causes the plurality of functional units placed in the container to communicate with each other.

2. The microphysiological system device according to claim 1, wherein a size of the container is equal to a size of a multi-well plate according to an industrial standard, as viewed in a front-back direction.

3. The microphysiological system device according to claim 1, wherein:
the container includes
a rectangular bottom wall including a pair of long-side portions disposed so as to face each other and a pair of short-side portions connecting the pair of long-side portions and disposed so as to face each other,
a pair of side walls standing from the pair of long-side portions toward the front side, and
a partition wall connecting the pair of side walls to each other, extending parallel to the short-side portion, and dividing a space between the pair of side walls into a plurality of container compartments; and
the container compartment is compatible with the plurality of functional units.

4. The microphysiological system device according to claim 1, wherein
the channel member includes a plurality of contact portions disposed so as to face the plurality of functional units placed in the container,
the contact portion contacts the functional unit from the front side, and
the channel is open to a back surface of the contact portion.

5. The microphysiological system device according to claim 1, wherein the channel member defines a plurality of the channels inside, and includes a flow rate control portion that controls a flow rate of a fluid flowing through a desired one of the channels.

6. The microphysiological system device according to claim 1, further comprising:
a cover member disposed on a front side of the channel member and having higher rigidity than the channel member; and
a fastening member that fastens the cover member and the container together, wherein the container and the channel member are combined by the cover member and the fastening member after the functional unit is fixed by the channel member.

7. The microphysiological system device according to claim 6, wherein
the cover member includes a front-side engaging portion,
the container includes a back-side engaging portion, and
the fastening member engages with the front-side engaging portion and the back-side engaging portion.

8. The microphysiological system device according to claim 7, wherein:
the container includes
a rectangular bottom wall including a pair of long-side portions disposed so as to face each other and a pair of short-side portions connecting the pair of long-side portions and disposed so as to face each other,
a pair of side walls standing from the pair of long-side portions toward the front side, and
a partition wall connecting the pair of side walls to each other, extending parallel to the short-side portion, and dividing a space between the pair of side walls into a plurality of container compartments;
a size of the container is equal to a size of a multi-well plate according to an industrial standard, as viewed in a front-back direction;
the container compartment is compatible with the plurality of functional units;
the channel member defines a plurality of the channels inside, and includes a flow rate control portion that controls a flow rate of a fluid flowing through a desired one of the channels, and a plurality of contact portions disposed so as to face the plurality of functional units placed in the container;
the contact portion contacts the functional unit from the front side, and the channel is open to a back surface of the contact portion; and
the container, the channel member, and the cover member are transparent.

9. The microphysiological system device according to claim 1, wherein
the container includes a bottom wall and a side wall standing from the bottom wall toward the front side, and
the side wall has an opening extending through the side wall.

10. The microphysiological system device according to claim 1, wherein
the container includes a bottom wall, and
the bottom wall has an opening extending through the bottom wall.

11. The microphysiological system device according to claim 1, wherein
the channel member includes a contact portion,
the functional unit has an opening,
the contact portion has a press-in portion that is pressed into the opening, and
the press-in portion is made of a material containing at least one of PDMS and resin.

12. The microphysiological system device according to claim 1, wherein the channel is used for degassing the functional unit.
